(19) United States
(12) Patent Application Publication
(10) Pub. No.: US 2025/0277280 A1
(43) Pub. Date: Sep. 4, 2025

(54) METHODS FOR SPATIALLY DETECTING RNA MOLECULES

(71) Applicant: CORNELL UNIVERSITY, Ithaca, NY (US)

(72) Inventors: David W. MCKELLAR, Ithaca, NY (US); Iwijn DE VLAMINCK, Ithaca, NY (US); Benjamin D. COSGROVE, Ithaca, NY (US); Madhav MANTRI, Ithaca, NY (US); Hao SHI, Ithaca, NY (US); loannis NTEKAS, Ithaca, NY (US)

(73) Assignee: CORNELL UNIVERSITY, Ithaca, NY (US)

(21) Appl. No.: 18/857,959

(22) PCT Filed: Apr. 19, 2023
(86) PCT No.: PCT/US2023/065929

§ 371 Date: Oct. 18, 2024

Related U.S. Application Data
(60) Provisional application No. 63/332,440, filed on Apr. 19, 2022.

(51) Int. Cl. C12Q 1/70; C12Q 1/6841; C12Q 1/6876

(52) U.S. Cl. CPC C12Q 1/701; C12Q 1/6841; C12Q 1/6876; C12Q 2600/158; C12Q 2600/16

(57) ABSTRACT

The present disclosure is directed to methods for enzymatic in situ polyadenylation of RNA enables detection of the full spectrum of RNAs, expanding the scope of sequencing-based spatial transcriptomics to the total transcriptome.

### CROSS REFERENCE TO RELATED APPLICATION

This application claims the benefit of U.S. Provisional Patent Application No. 63/332,440, filed Apr. 19, 2022, the contents of which are incorporated herein by reference in its entirety.

### INCORPORATION BY REFERENCE OF SEQUENCE LISTING

The sequence listing in the XML, named as 40790_10252_02_PC_SequenceListing.xml of 4 KB, created on Apr. 18, 2023, and submitted to the United States Patent and Trademark Office via Patent Center, is incorporated herein by reference.

### BACKROUND

Spatial transcriptomics provides insight into the spatial context of gene expression (Rao, A., et al., Exploring tissue architecture using spatial transcriptomics. Nature vol. 596 211-220 (2021); Marx, V. Method of the Year: spatially resolved transcriptomics. Nature Methods 18, 9-14 (2021)). Current methods are restricted to capturing polyadenylated transcripts and are not sensitive to many species of non-A-tailed RNAs, including microRNAs, newly transcribed RNAs, and non-host RNAs. Extending the scope of spatial transcriptomics to the total transcriptome would enable observation of spatial distributions of regulatory RNAs and their targets, link non-host RNAs and host transcriptional responses, and deepen our understanding of cell-cell interactions and spatial biology.

Current strategies for spatial transcriptomics broadly fall into two categories, in-situ-hybridization-based (ISH) methods and sequencing-based methods (Rao, A., et al., Exploring tissue architecture using spatial transcriptomics. Nature vol. 596 211-220 (2021). ISH methods are targeted and require the design of complex pools of oligonucleotide probes to assay a defined set of RNAs3,4. The pool of targets is only limited by target sequence uniqueness and length. These criteria exclude many small RNAs and mean that ISH methods are not sensitive to post-transcriptional modifications like splicing, unless they are specifically built into the probe set. Most importantly, targeted ISH methods cannot be used to discover new RNAs. This limitation is especially prevalent in the context of infectious disease and the microbiome where the species present are often unknown prior to the experiment.

Most sequencing-based methods rely on endogenous poly(A) tails added after transcription for capture of RNA and use DNA sequencing to count molecules (Rodriques, S. G. et al. Slide-seq: A scalable technology for measuring genome-wide expression at high spatial resolution. Science (1979) 363, 1463-1467 (2019); Stickels, R. R. et al. Highly sensitive spatial transcriptomics at near-cellular resolution with Slide-seqV2. Nature Biotechnology 1-7 (2020) doi: 10.1038/s41587-020-0739-1). This chemistry enables broad capture of messenger RNAs and a portion of long noncoding RNAs. We and others have also shown that non-A-tailed molecules may be spuriously captured at a much lower rate, but only if the sequence has an A-rich region. One major strength of sequencing-based strategies is their flexibility for discovery. Whereas ISH methods rely on reference genomes and gene annotations to design probes, sequencing methods paired with bioinformatics tools can be used to detect unknown genes and even molecules derived from non-eukaryotic sources. However, these methods are mostly limited to capturing endogenously polyadenylated RNAs.

Methods for bulk total RNA sequencing have used adapter ligation or polyadenylation of free RNA to circumvent these limitations (Yang, X. et al. PALM-Seq: integrated sequencing of cell-free long RNA and small RNA. doi: 10.1101/686055). Unfortunately, these chemistries have proven difficult to translate into single-cell and spatial transcriptomics. Random hexamer-based chemistries, which use random oligonucleotides for capture rather than poly(dT) sequences, have been adapted and even commercialized (see Parse Biosciences), but these methods introduce substantial biases which reduce the utility of the resulting datasets. Currently, single-cell RNA-sequencing methods for the capture of non-A-tailed RNAs require targeted probe design, have low throughput, or require custom microfluidic devices (Saikia, M. et al., Simultaneous multiplexed amplicon sequencing and transcriptome profiling in single cells. Nat Methods 16, 59-62 (2019); Verboom, K. et al. SMARTer single cell total RNA sequencing. bioRxiv 430090 (2018) doi: 10.1101/430090, Isakova, A., et al., Single-cell quantification of a broad RNA spectrum reveals unique noncoding patterns associated with cell types and states. Proceedings of the National Academy of Sciences 118, e2113568118 (2021); Salmen, F. et al. Droplet-based Single-cell Total RNA-seq Reveals Differential Non-Coding Expression and Splicing Patterns during Mouse Development. bioRxiv 2021.09.15.460240 (2021) doi: 10.1101/2021.09.15.460240).

### SUMMARY

The current disclosure is directed to methods for spatial detection of RNA molecules in a biological sample. The method described herein advantageously possesses the ability to spatially detect all types of RNAs regardless of the length of the RNAs and whether they are coding or noncoding RNAs.

One aspect of the current disclosure is directed to a method for spatial detection of RNA molecules in a biological sample, comprising:
(a) providing a substrate defined by an array of spots, wherein each spot comprises DNA oligomers immobilized thereto, wherein each of the DNA oligomers comprises:
   (i) a spatial barcode, wherein all DNA oligomers in one spot share a same spatial barcode, which is different from the spatial barcodes in other spots; and
   (ii) a poly(dT) sequence;
(b) placing a biological sample onto the substrate;
(c) contacting the substrate with a Poly(A) polymerase enzyme mix comprising a Poly(A) polymerase and a polymerase reaction buffer reagent to perform in situ polyadenylation;
(d) capturing RNA molecules from the biological sample;
(e) adding reverse transcription reagents to generate cDNA molecules from captured RNA molecules, wherein cDNA molecules generated from RNAs captured by DNA oligomers on a spot comprise a spatial barcode common to the spot; and
(f) obtaining a map of spatial gene expression based on the cDNA molecules generated.

In some embodiments, each of the DNA oligomers comprises an oligonucleotide sequence and/or a unique molecular identifier sequence. In some embodiments, the substrate is composed of a material selected from the group consisting of glass, silicon, poly-L-lysine coated material, nitrocellulose, polystyrene, cyclic olefin copolymers (COCs), cyclic olefin polymers (COPs), polypropylene, polyethylene and polycarbonate. In some embodiments, the array of spots comprises 10-100,000,000 spots. In some embodiments, the array of spots comprises at least 10, at least 100, at least 1,000, at least 10,000, at least 50,000, at least 100,000, at least 200,000, at least 300,000, at least 400,000, at least 500,000, at least 1,000,000, at least 2,000,000, at least 5,000,000, at least 10,000,000, at least, 20,000,000, at least 30,000,000, at least 40,000,000, at least 50,000,000, at least 75,000,000, or at least 100,000,000 spots. In some embodiments of the disclosure, the array is placed within a capture area in the range of about 1 mm² to about 100 mm². In some embodiments, the capture area is of a dimension of up to 100 mm². In some embodiments, the capture area has a dimension of up to 75 mm². In some embodiments, the capture area has a dimension of up to 50 mm². In some embodiments, the capture area has a dimension of up to 25 mm². In some embodiments, the capture area has a dimension of up to 15 mm². In some embodiments, the capture area has a dimension of up to 10mm². In some embodiments, the capture area has a dimension of up to 6.5 mm². In some embodiments, the capture area has a dimension of up to 3 mm². In some embodiments, the substrate comprises multiple capture areas, each comprising an array of spots. Generally, the spots within an array or arrays on the same substrate have the substantially same size. However, spots of different arrays or substrates can differ size. In some embodiments, the spots are about 10 nm to about 1 mm in diameter. In some embodiments, the spots are about 100 nm to 1 mm in diameter. In some embodiments, the spots are about 1 μm to 1 mm in diameter. In some embodiments, the spots are about 150 nm to about 70 μm in diameter. In some embodiments, the spots are about 1 μm to about 100 μm in diameter. In some embodiments, the spots are about 1 μm to about 40 μm in diameter. In some embodiments, the spots are about 10 μm to about 40 μm in diameter. In some embodiments, the spots are about 50 μm to about 70 μm in diameter. In some embodiments, the spots are about 500 nm to about 125 μm apart as measured by center of spot to center of spot. In some embodiments, the spots are up to 60 μm in diameter and are no more than 100 μm apart as measured by center of spot to center of spot. In some embodiments, the spots are up to 220 nm in diameter and are no more than 750 nm apart as measured by center of spot to center of spot. In some embodiments, the spots are in an organized pattern in the array. In some embodiments, the spots are randomly distributed in within an array. In some embodiments, the spatial resolution of the array can range from about 10 nm to about 1 mm. In some embodiments, the spatial resolution of the array can range from about 1 micron to about 100 microns, or about 5 microns to about 75 microns. In some embodiments, the spatial resolution of the array can range from about 1 micron to about 100 microns, or about 5 microns to about 75 microns. In some embodiments, the spatial resolution is about 60 microns. In some embodiments, the spatial resolution is about 10 microns. In some embodiments, the spatial resolution is less than 10 microns and at a subcellular level, e.g., about 5 micros, or about 1 micron.

In some embodiments, the disclosure is directed to methods for spatial detection of RNA molecules in a biological sample where step (b) further comprises fixing the biological sample (e.g., using formaldehyde, Formalin-fixed, parafin-embedded (FFPE), Acetone, Methanol+acetone, Glyoxal fixation, or methacarn fixation). In some embodiments, step (b) further comprises staining the fixed biological sample. In some embodiments, step (b) further comprises capturing an image of the fixed and stained biological sample.

In some embodiments of the current disclosure, step (c) further comprises, prior to the contacting step, equilibrating the substrate by adding a wash buffer comprising Poly(A) polymerase reaction buffer, an RNase inhibitor, and nuclease free water to the substrate. In some embodiments, step (c) comprises, after the equilibrating, adding a Poly(A) polymerase enzyme mix which comprises Poly(A) polymerase reaction buffer, a Poly(A) polymerase enzyme, adenosine triphosphate (ATP), RNase inhibitor, and nuclease-free water and incubating. In some embodiments, the Poly(A) polymerase enzyme is a yeast Poly(A) polymerase.

In some embodiments of the disclosure, step (d) includes permeabilizing the cells in the biological sample to permit release and capture of the RNA molecules from the cells in the biological sample. In some embodiments of the disclosure, step (e) further comprises initiating second strand synthesis via the addition of a second strand primer.

In some embodiments, the sequences of the generated cDNA are obtained. In some embodiments, the generated cDNAs with spatial barcodes and the cDNA sequences are used to map the spatial gene expression. In some embodiments, the generated cDNAs and the cDNA sequences are correlated with the captured image of the fixed and stained biological sample to map the spatial gene expression. In some embodiments, the cDNAs are denatured and transferred from the spots to a solution readily usable for amplification. In some embodiments, the amplified cDNAs are further processed for optimal amplicon size.

Some embodiments of the disclosure are directed to methods for spatial detection of RNA molecules in a biological sample where the length of the poly(A) tail is controlled in the in situ polyadenylation step. In some embodiments, the length of the poly(A) tail is about 10 base pairs to about 4,000 base pairs. In some embodiments, the length of the poly(A) tail is less than about 2,000 base pairs. In some embodiments, the length of the poly(A) tail is less than about 1,600 base pairs. In some embodiments, the length of the poly(A) tail is less than about 1,000 base pairs. In some embodiments, the Poly(A) polymerase enzyme mix includes (i) ATP and (ii) biotin-11-ATP or dATP at a ratio of at least 5:1. In some embodiments, the ratio of ATP to biotin-11-ATP or dATP is 1:1. In some embodiments, the poly(dT) sequence comprises a VN sequence at the 3′ end wherein the V is any nucleotide base other than T and Nis any nucleotide base.

Some embodiments of the discourse are directed to methods for spatial detection of RNA molecules in a biological sample where the biological sample is a tissue. In some embodiments, the tissue is selected from the group of connective tissue, epithelial tissue, muscle tissue, and nervous tissue. In some embodiments, the muscle tissue is a cardiac, skeletal, or smooth muscle tissue. In some embodiments, the epithelial tissue is simple squamous, stratified squamous, simple cuboidal, stratified cuboidal, simple columnar, stratified columnar, pseudostratified columnar, or transitional epithelia. In some embodiments, the connective tissue is connective tissue proper or specialized connective tissue. In some embodiments, the connective tissue proper is loose or dense tissue, comprising collagen, reticular, or elastic fibers. In some embodiments, the specialized connective tissue comprises adipose, cartilage, bone, blood, reticular, and lymphatic tissues. In some embodiments, the biological sample is a combination of tissue types which form an organ. In some embodiments, the biological sample is taken from a testis. In some embodiments, the biological sample is a histological section of tissue. In some embodiments, the biological sample is a tissue sample of an injured tissue, or an organ suspected to suffer an infection. In some embodiments, the tissue sample is a tumor section, gut microbiome, brain section, patient biopsy, or a plant sample. In some embodiments, the method further comprises comparing the spatial gene expression map of the tissue sample to (i) the spatial gene expression map of a control sample, or (ii) the spatial gene expression map of another sample of the same tissue taken at a different time point.

Some embodiments of the disclosure are directed to methods for spatial detection of RNA molecules in a biological sample where the RNAs captured are ribonucleic acids (RNAs), RNA degradation products, RNAs comprising a poly(A) tail, messenger RNA (mRNA), long noncoding RNAs (lncRNAs), long intergenic noncoding RNAs (lincRNAs), cis-natural antisense transcripts (cisNATs), antisense RNAs, ribosomal RNAs (rRNAs), microRNAs (miRNAs), small interfering RNAs (siRNAs), short hairpin RNAs (shRNAs), guide RNAs (gRNAs), transfer RNAs (tRNAs), small nuclear RNAs (snRNAs), small nucleolar RNAs (snoRNAs), small Cajal body-specific RNA (scaRNAs), enhancer RNAs (eRNAs), piwi-interacting RNAs (piRNAs), Y RNAs, non-coding RNAs, vault RNA, viral RNA, microbial RNA such as bacterial RNA, archaeal RNA, or fungal RNA, or combinations thereof. In some embodiments, the RNAs captured are viral RNA, bacterial RNA, archaeal RNA, fungal RNA, or a combination thereof.

Some embodiments of the disclosure further comprise isolating a subpopulation of cDNAs from the cDNAs generated in step (e). In some embodiments, the subpopulation of cDNAs is generated from viral RNAs, bacterial RNA, archaeal RNA, or fungal RNA. Some embodiments of the disclosure further comprise obtaining the sequences of the cDNAs in the isolated subpopulation.

Another aspect of the current disclosure is directed to a kit comprising a substrate defined by an array of spots, wherein each spot comprises DNA oligomers immobilized on the substrate, at least one reagent comprising a Poly(A) polymerase enzyme mix; and, optionally, instructions for use. In some embodiments, each of the DNA oligomers comprises: (i) a spatial barcode, wherein all primers in one spot share the same spatial barcode, which is different from the spatial barcodes in other spots; and (ii) a poly(dT) sequence. In some embodiments, each of the DNA oligomers further comprises: an oligonucleotide sequence; and/or a unique molecular identifier sequence. In some embodiments, the Poly(A) polymerase enzyme mix comprises a polymerase reaction buffer reagent; a poly(A) polymerase enzyme reagent; and, optionally, nuclease free water reagent. In some embodiments, the Poly(A) polymerase enzyme mix further comprises adenosine triphosphate reagent and/or an RNase inhibitor reagent. In some embodiments, the kit further comprises a wash buffer reagent. In some embodiments, the wash buffer reagent comprises: a polymerase reaction buffer reagent, an RNase inhibitor reagent; and, optionally, a nuclease-free water reagent. In some embodiments, the Poly(A) polymerase enzyme mix comprises (i) ATP and (ii) biotin-11-ATP or dATP, optionally at a ratio that is greater than about 5:1 ATP to biotin-11-ATP or dATP. In some embodiments, the poly(dT) sequence comprises a VN sequence at the 3′ end wherein the V is any nucleotide base other than T and N is any nucleotide base. In some embodiments, the kit comprises reagents that are either ready to use, concentrated, or a combination of ready to use and concentrated. In some embodiments, the reagents are provided in separate containers or provided in pre-mixed quantities of any combination of reagents. In some embodiments, the array of spots comprises 10-100,000,000 spots, such as at least 10, at least 100, at least 1,000, at least 10,000, at least 50,000, at least 100,000, at least 200,000, at least 300,000, at least 400,000, at least 500,000, at least 1,000,000, at least 2,000,000, at least 5,000,000, at least 10,000,000, at least, 20,000,000, at least 30,000,000, at least 40,000,000, at least 50,000,000, at least 75,000,000, or at least 100,000,000 spots. In some embodiments, the array is placed within a capture area in the range of about 1 mm² to about 100 mm². In some embodiments, the capture area has a dimension of up to 75 mm². In some embodiments, the capture area has a dimension of up to 50 mm². In some embodiments, the capture area has a dimension of up to 25 mm². In some embodiments, the capture area has a dimension of up to 15 mm². In some embodiments, the capture area has a dimension of up to 10 mm². In some embodiments, the capture area has a dimension of up to 6.5 mm². In some embodiments, the capture area has a dimension of up to 3 mm². In some embodiments, the substrate comprises multiple capture areas, each comprising an array of spots. Generally, the spots within an array or arrays on the same substrate have the substantially same size. However, spots of different arrays or substrates can differ size. In some embodiments, the spots are about 10 nm to about 1 mm in diameter. In some embodiments, the spots are about 100 nm to 1 mm in diameter. In some embodiments, the spots are about 1 μm to 1 mm in diameter. In some embodiments, the spots are about 150 nm to about 70 μm in diameter. In some embodiments, the spots are about 1 μm to about 100 μm in diameter. In some embodiments, the spots are about 1 μm to about 40 μm in diameter. In some embodiments, the spots are about 10 um to about 40 μm in diameter. In some embodiments, the spots are about 50 μm to about 70 um in diameter. In some embodiments, the spots are about 500 nm to about 125 μm apart as measured by center of spot to center of spot. In some embodiments, the spots are up to 60 μm in diameter and are no more than 100 μm apart as measured by center of spot to center of spot. In some embodiments, the spots are up to 220 nm in diameter and are no more than 750 nm apart as measured by center of spot to center of spot. In some embodiments, the spots are in an organized pattern in the array. In some embodiments, the spots are randomly distributed in within an array. In some embodiments, the spatial resolution of the array can range from about 10 nm to about 1 mm. In some embodiments, the spatial resolution of the array can range from about 1 micron to about 100 microns, or about 5 microns to about 75 microns. In some embodiments, the spatial resolution of the array can range from about 1 micron to about 100 microns, or about 5 microns to about 75 microns. In some embodiments, the spatial resolution is about 60 microns. In some embodiments, the spatial resolution is about 10 microns. In some embodiments, the spatial resolution is less than 10 microns and at a subcellular level, e.g., about 5 micros, or about 1 micron.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1A-G is a representation of the steps involved in the Visium Spatial Gene Expression protocol. (A) shows the Visium Spatial Gene Expression Slide and its capture areas. (B) shows the tissue staining and imaging of step 1. (C) represents the permeabilization of step 2. (D) represents step 3. (E) represents the cDNA amplification of step 4. (F) shows the amplified cDNA processing included in step 5. and (G) shows the sequencing of step 6.

FIG. 2A-D shows the in situ poly(A) tailing in mouse gut tissue. E. coli poly(A) polymerase enables in situ poly(A) tailing of transcripts in mouse gut tissue. A fluorescent probe which broadly targets microbial 16S ribosomal RNAs (Eub) is shown in pink to label microbes. A poly(T) fluorescent probe which labels poly(A) tails is shown in blue. (A), the upper left panel, is negative control, where no poly(A) polymerase was used. (B), (C), and (D) show multiple fields of views where microbial transcripts are poly(A) tailed by E. coli poly(A) polymerase and detected by poly(T) fluorescent probes.

FIG. 3A-F shows in situ polyadenylation enables spatial profiling of noncoding and non-host RNAs. (A) Workflow for Spatial Total RNA-Sequencing (STRS). (B) Comparison of select RNA biotypes between Visium and STRS datasets. Y-axis shows the percent of unique molecules (UMIs) for each spot. (C) Detection of coding and noncoding RNAs between Visium and STRS workflows. Color scale shows average log-normalized UMI counts. Dot size shows the percent of spots in which each RNA was detected. (D) Log 10-transformed coverage of deduplicated reads mapping to sense (light gray) and antisense (dark gray) strands at the Vaultrc5, ENSMUSG00002075551, and Rps8 loci. Annotations shown are from GENCODE M28 and include one of the five isoforms for Rps8 as well as the four intragenic features within introns of Rps8. (E) Spatial maps of coding and noncoding transcripts for Visium and STRS workflows. Spots in which the transcript was not detected are shown as gray. (F) Detection of reovirus transcripts using the standard workflow, STRS, and STRS with targeted pulldown enrichment. Spots in which the virus was not detected are shown as gray.

FIG. 4A-E is a comparison of bioinformatic analyses for Visium and Spatial Total RNA-Sequencing (STRS). (A) Bioinformatic tools and workflows used to preprocess, align, and quantitate transcripts. (B) STAR alignment rate for reads mapping to unique genomic position (x-axis) versus reads uniquely mapping to annotated regions (GENCODE M28 annotations) of the genome (y-axis). Each point represents an entire Visium capture area. Points are colored by sample preparation method (see Methods) and are shaped according to tissue type. (C) STAR alignment rate for reads mapping to unique or multiple positions along the genome (x-axis) versus reads uniquely mapping or multimapping to annotated regions (GENCODE M28 annotations) of the genome (y-axis). Spots are colored as in (B). (D) Number of unique molecules (UMIs) detected by STARsolo (x-axis) versus kallisto (y-axis). Each point represents a barcoded spot. Points are colored by sample preparation method and are shaped according to tissue type. (E) Number of features detected by STARsolo (x-axis) versus kallisto (y-axis). Spots are colored as in (D).

FIG. 5A-D is a gene-by-gene comparison across Visium and Spatial Total RNA-Sequencing (STRS). Genes are split between non-protein coding (A and C) and protein-coding (B and D) genes. Data is shown for injured skeletal muscle at 2 days post-injury (A and B) and infected heart samples (C and D).

FIG. 6 is a transcript biotype spatial distribution comparison between Visium and Spatial Total RNA-Sequencing (STRS) for regenerating mouse skeletal muscle. Color scale shows the percent of unique molecules (UMIs) for each spot that correspond to each transcript biotype. Gray spots contain no molecules which correspond to the given biotype. Transcript biotypes shown include protein coding, ribosomal RNA (rRNA), mitochondrial ribosomal RNA (Mt_rRNA), microRNA (miRNA), long noncoding RNAs (lncRNA), mitochondrial transfer RNAs (Mt_tRNA), small nucleolar RNA (snoRNA), small nuclear RNA (snRNA), ribozyme, miscellaneous RNA (misc_RNA), and small Cajal body-specific RNA (scaRNA).

FIG. 7 is a transcript biotype spatial distribution comparison between Visium and Spatial Total RNA-Sequencing (STRS) for mouse hearts with and without Reovirus infection. Color scale shows the percent of unique molecules (UMIs) for each spot that correspond to each transcript biotype. Gray spots contain no molecules which correspond to the given biotype. Transcript biotypes shown include protein coding, ribosomal RNA (rRNA), mitochondrial ribosomal RNA (Mt rRNA), microRNA (miRNA), long noncoding RNAs (lncRNA), mitochondrial transfer RNAs (Mt_tRNA), small nucleolar RNA (snoRNA), small nuclear RNA (snRNA), ribozyme, miscellaneous RNA (misc_RNA), and small Cajal body-specific RNA (scaRNA).

FIG. 8A-F shows Spatial total RNA-sequencing of regenerating skeletal muscle (A) H&E histology of mouse tibialis anterior muscles collected 2-, 5-, and 7-days post-injury (dpi). (B) Clustering of spot transcriptomes based on total transcriptome repertoires (see Methods). (C) Differentially expressed RNAs across regional clusters. Y-axis shows log-normalized expression of each feature. Mean expression across each cluster is reported, colored according to the legend in (B). Error bars show standard deviation. Reported statistics to the right of plots reflect differential gene expression analysis performed across clusters on merged STRS samples (Wilcoxon, see Methods). Asterisks next to transcript names reflect differential expression analysis performed across skeletal muscle Visium and STRS samples (**p_val_adj<10-50, ***p_val_adj<10-150; Wilcoxon, see Methods). (D) Spatial maps for select features from (C). (E) Mature miRNA expression detected by STRS. Color scale shows log-normalized miRNA counts, quantified by miRge3.0 (Methods). (F) Average detection of miRNAs compared between small RNA-sequencing (n=8) and STRS (n=4). Axes show log2 counts per million transcripts, normalized to the total number of transcripts which map to small RNA loci with miRge3.0. The top 100 most abundant miRNAs detected by smRNAseq are shown.

FIG. 9A-B is a comparison of mature microRNA detection in small RNA-sequencing, Visium, and Spatial Total RNA-Sequencing. Counts for (A) heart samples and (B) skeletal muscle samples are shown as log 2-transformed counts per million (CPM) with a pseudocount of 1. Counts reflect UMI-deduplicated reads for STRS samples and are normalized to the total number of counts which align to mature microRNAs.

FIG. 10A-E shows STRS enables simultaneous analysis of viral infection and host response. (A) H&E staining of mock and reovirus-infected hearts collected using the standard Visium workflow and STRS. (B) Tissue regions identified through unsupervised clustering of spot transcriptomes. Color legend is shown in (D). (C) Log-normalized expression of noncoding and coding RNAs which are highly expressed in myocarditic regions. Spots in which transcripts were not detected are shown in gray. (D) Normalized coverage of deduplicated reads for the sense [+] and antisense [−] strands of all ten reovirus gene segments. X-axis shows the length-normalized position across the gene bodies of all ten reovirus segments. Note that the peak in antisense [−] coverage for the Visium sample (blue) corresponds to only 11 total reads. (E) Co-expression of pulldown-enriched reovirus UMIs versus infection-associated genes.

FIG. 11A-B shows the STRS used with Curio Seeker protocol dubbed STRS-HD. Mice were orally infected with type 1-lang Reovirus, and heart tissues were collected seven days post-infection. (A) Spatial map showing the capture of RNAs which map to the host genome or to the reovirus genome. The top row shows the tissue processed with the standard Seeker workflow. The bottom row shows the tissue processed using STRS-HD. (B) Zoomed in view of a region with high reovirus capture.

FIG. 12A-I. Shows comparisons of several transcript biotypes shown in testis tissue as performed in Seeker and in STRS-HD. A) is the H&E stained 3 mm-by-3 mm square of testis showing the area captured in the Seeker data where the scale bar is 1000 μm. B) shows protein coding RNA in both Seeker (top row) and STRS-HD (bottom row). C) shows long noncoding (lncRNA) in both Seeker (top row) and STRS-HD (bottom row). D) shows miscellaneous RNA (miscRNA) in both Seeker (top row) and STRS-HD (bottom row). E) shows microRNA (miRNA) in both Seeker (top row) and STRS-HD (bottom row). F) shows transfer RNAs (tRNAs) in both Seeker (top row) and STRS-HD (bottom row). G) shows small nucleolar RNAs (snoRNAs) in both Seeker (top row) and STRS-HD (bottom row). H) shows small nuclear RNAs (snRNAs) in both Secker (top row) and STRS-HD (bottom row). I) shows ribozyme in both Seeker (top row) and STRS-HD (bottom row).

FIG. 13 Tuning poly(A) tail length via biotin-11-ATP. Purified transfer RNA (120 bp long, pink) was incubated with yeast poly(A) polymerase with varying ratios of ATP to biotin-11-ATP (B-11-ATP). Total concentration of ATP+B-11-ATP was held constant across experimental conditions. The x-axis shows the lengths of RNAs after polyadenylation, and the y-axis shows the abundance of RNAs, normalized by sample. Reactions were performed to match the conditions of STRS.

FIG. 14A-D. Murine gut samples were sectioned and processed using either Visium (A and B) or STRS (C and D). Two gut sections were placed in each capture area, and are outlined in red. Spatial maps show the number of unique molecules (UMIs) which map to microbial genomes are detected in each spot (A and C) and the number of total microbial taxa are detected in each spot (B and D).

### DETAILED DESCRIPTION

Although claimed subject matter will be described in terms of certain examples, other examples, including examples that do not provide all the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value).

“Complementary” or “substantially complementary” refers to the hybridization or base pairing or the formation of a duplex between nucleotides or nucleic acids, such as, for instance, between the two strands of a double-stranded DNA molecule or between an oligonucleotide primer and a primer binding site on a single-stranded nucleic acid. Complementary nucleotides are, generally, A and T (or A and U), or C and G. Two single-stranded RNA or DNA molecules are said to be substantially complementary when the nucleotides of one strand, optimally aligned and compared and with appropriate nucleotide insertions or deletions, pair with at least about 80% of the other strand, usually at least about 90% to about 95% (e.g., at least 90%, at least 91%, at least 92%, at least 93%, at least 94%, and at least 95%), and even at least about 98% to about 100% (e.g., at least 98%, at least 99%, and 100%).

“Hybridization” refers to the process in which two single-stranded polynucleotides bind non-covalently to form a stable double-stranded polynucleotide. The resulting (usually) double-stranded polynucleotide is a “hybrid” or “duplex.” “Hybridization conditions” will typically include salt concentrations of approximately up to 1M, often up to about 500 mM and may be up to about 200 mM. A “hybridization buffer” is a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are often performed under stringent conditions, i.e., conditions under which a primer will hybridize to its target subsequence but will not hybridize to the other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tm for the specific sequence at a defined ionic strength and pH. Exemplary stringent conditions include a salt concentration of at least 0.01 M to no more than 1M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized.

“Nucleic acid”, “oligonucleotide”, “oligo” or grammatical equivalents used herein refers generally to at least two nucleotides covalently linked together. A nucleic acid generally will contain phosphodiester bonds, although in some cases nucleic acid analogs may be included that have alternative backbones such as phosphoramidite, phosphorodithioate, or methylphophoroamidite linkages; or peptide nucleic acid backbones and linkages. Other analog nucleic acids include those with bicyclic structures including locked nucleic acids, positive backbones, non-ionic backbones and non-ribose backbones. Modifications of the ribose-phosphate backbone may be done to increase the stability of the molecules; for example, PNA: DNA hybrids can exhibit higher stability in some environments.

“Primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Sequencing”, “sequence determination” and the like means determination of information relating to the nucleotide base sequence of a nucleic acid. Such information may include the identification or determination of partial as well as full sequence information of the nucleic acid. Sequence information may be determined “with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, i.e. where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technology, Inc., Carlsbad, Calif.); sequencing by synthesis using modified nucleotides (such as commercialized in TruSeq™ and HiSeg™ technology by Illumina, Inc., San Diego, Calif., HeliScope™ by Helicos Biosciences Corporation, Cambridge, Mass., and PacBio RS by Pacific Biosciences of California, Inc., Menlo Park, Calif.), sequencing by ion detection technologies (Ion Torrent, Inc., South San Francisco, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

One aspect of the current disclosure is directed to a method for spatial detection of RNA molecules in a biological sample, including both coding and noncoding RNA molecules in a biological sample. The present method typically comprises the steps of: (a) providing a substrate defined by an array of spots, wherein each spot comprises DNA oligomers immobilized on the substrate, wherein each of the DNA oligomers comprises: (i) a spatial barcode, wherein all DNA oligomers in one spot share the same spatial barcode, which is different from the spatial barcodes in other spots; and (ii) a poly(dT) sequence; (b) placing a biological sample onto the substrate; (c) contacting the substrate with a Poly(A) polymerase enzyme mix comprising a Poly(A) polymerase and a polymerase reaction buffer reagent to perform in situ polyadenylation; (d) capturing RNA molecules from the biological sample; (e) adding reverse transcription reagents to generate cDNA molecules from captured RNA molecules, wherein cDNA molecules generated from RNAs captured by DNA oligomers on a spot comprise a spatial barcode common to the spot; and (f) obtaining a map of spatial gene expression based on the cDNA molecules generated.

### The Array

In some embodiments, the method comprises the use of a substrate comprising an array of spots, wherein each spot comprises DNA oligomers immobilized on the substrate. In some embodiments, the substrate is a solid, planar, and/or rigid substrate or support which is insoluble in aqueous liquid. The substrate can be non-porous or porous. The substrate can optionally be capable of taking up a liquid (e.g. due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers. Particularly useful solid supports for some embodiments are slides.

In some embodiments, the substrate is a solid support composed of a material selected from the group consisting of glass, silicon, poly-L-lysine coated material, nitrocellulose, polystyrene, cyclic olefin copolymers (COCs), cyclic olefin polymers (COPs), polypropylene, polyethylene and polycarbonate.

In some embodiments, the substrate comprises at least one array, where each array comprises a plurality of spots. As used herein, “spots” refer to areas on the substrate where DNA oligomers are immobilized to the substrate. In some embodiments, the DNA oligomers are immobilized to the substrate directly. In some embodiments, the DNA oligomers are immobilized to the substrate indirectly. In embodiments where the DNA oligomers are immobilized to the substrate indirectly, such indirect immobilization can occur through beads or other particles to which the oligomers attach. In some embodiments, one bead is present for each spot within an array. In some embodiments, multiple beads are present at each spot within an array.

The spots of an array can be randomly spaced such that nearest neighboring spots have variable spacing between each other. Alternatively, the spacing between spots on the array can be ordered, for example, forming a regular pattern such as a rectilinear grid or hexagonal grid.

The spots of the array can be in any shape. In some embodiments, the spots within an array are generally in the same or similar shape. In some embodiments, the spots are circular. In some embodiments, the spots are any type of polygon. In some embodiments, the spots are triangular. In some embodiments, the spots are quadrilaterals. In some embodiments, the spots are pentangular. In some embodiments, the spots are hexagons. In some embodiments, the spots are heptagons. In some embodiments, the spots are octagons. In some embodiments, the spots are nonagons. In some embodiments, the spots are decagons.

In some embodiments, the spots are about 10 nm to about 1 mm in diameter. In some embodiments, the spots are about 100 nm to about 1 mm in diameter. In some embodiments, the spots are about 1 μm to about 1 mm in diameter. In some embodiments, the spots are about 100 nm to about 500 μm in diameter. In some embodiments, the spots are about 115 nm to about 250 μm in diameter. In some embodiments, the spots are about 125 nm to about 175 μm in diameter. In some embodiments, the spots are about 130 nm to about 100 μm in diameter. In some embodiments, the spots in various arrays can range from about 150 nm to about 70 μm in diameter. Generally, the spots within one array are of substantially the same size. In some embodiments, the spots are between about 200 nm and about 65 μm in diameter. In some embodiments, the spots are between about 220 nm and about 60 μm in diameter. In some embodiments, the spots are about 1 μm to about 100 μm in diameter. In some embodiments, the spots are about 1 μm to about 40 μm in diameter. In some embodiments, the spots are about 10 μm to about 40 μm in diameter. In some embodiments, the spots are about 50 μm to about 70 μm in diameter. In some embodiments, the spots are no larger than about 70 μm in diameter. In some embodiments, the spots are about 60 μm in diameter. In some embodiments, the spots are about 50 μm in diameter. In some embodiments, the spots are about 40 μm in diameter. In some embodiments, the spots are about 30 μm in diameter. In some embodiments, the spots are about 20 μm in diameter. In some embodiments, the spots are about 10 μm in diameter. In some embodiments, the spots are about 1 μm in diameter. In some embodiments, the spots are about 950 nm in diameter. In some embodiments, the spots are about 900 nm in diameter. In some embodiments, the spots are about 850 nm in diameter. In some embodiments, the spots are about 800 nm in diameter. In some embodiments, the spots are about 750 nm in diameter. In some embodiments, the spots are about 700 nm in diameter. In some embodiments, the spots are about 650 nm in diameter. In some embodiments, the spots are about 600 nm in diameter. In some embodiments, the spots are about 550 nm in diameter. In some embodiments, the spots are about 400 nm in diameter. In some embodiments, the spots are about 350 nm in diameter. In some embodiments, the spots are about 300 nm in diameter. In some embodiments, the spots are no larger than 250 nm in diameter. In some embodiments, the spots are about 200 nm in diameter. In some embodiments, the spots are about 150 nm in diameter.

In some embodiments, the array of spots comprises as few as 10 spots and up to 100,000,000 spots. In some embodiments, the array comprises at least 10 spots. In some embodiments the array comprises at least 100 spots. In some embodiments, the array comprises at least 1,000 spots. In some embodiments, the array comprises at least 10,000 spots. In some embodiments, the array comprises at least 50,000 spots. In some embodiments, the array comprises at least 100,000 spots. In some embodiments, the array comprises at least 200,000 spots. In some embodiments, the array comprises at least 300,000 spots. In some embodiments, the array comprises at least 400,000 spots. In some embodiments, the array comprises at least 500,000 spots. In some embodiments, the array comprises at least 1,000,000 spots. In some embodiments, the array comprises at least 2,000,000 spots. In some embodiments, the array comprises at least 10,000,000 spots. In some embodiments, the array comprises at least 15,000,000 spots. In some embodiments, the array comprises at least 20,000,000 spots. In some embodiments, the array comprises at least 30,000,000. In some embodiments, the array comprises at least 40,000,000 spots. In some embodiments, the array comprises at least 50,000,000 spots. In some embodiments, the array comprises at least 75,000,000 spots. In some embodiments, the array comprises at least 100,000,000 spots. In some embodiments, the array comprises any range of spots between 10 and any number up to and including 75,000,000 spots. In some embodiments, the array comprises 10 to 1,000 spots.

In some embodiments, beads are used in the array to indirectly bind the DNA oligomers. As used herein, “beads”, “microbeads”, “microspheres” or “particles” or grammatical equivalents can include small discrete particles. The composition of the beads can vary, depending upon the class of capture probe, the method of synthesis, and other factors. Suitable bead compositions include those used in peptide, nucleic acid and organic moiety synthesis, including, but not limited to, plastics, ceramics, glass, polystyrene, methylstyrene, acrylic polymers, paramagnetic materials, thoriasol, carbon graphite, titanium dioxide, latex or cross-linked dextrans such as Sepharose, cellulose, nylon, cross-linked micelles and Teflon may all be used. “Microsphere Detection Guide” from Bangs Laboratories, Fishers IN is a helpful guide, which is incorporated herein by reference in its entirety. The beads need not be spherical; irregular particles may be used. In addition, the beads may be porous, thus increasing the surface area of the bead available for either capture probe attachment or tag attachment. The size of the beads can range from nanometers, for example, 100 nm, to millimeters, for example, 1 mm, with beads from about 0.2 μm to about 200 μm commonly employed, and from about 5 μm to about 20 μm being within the range currently exemplified, although in some embodiments smaller or larger beads may be used. In certain embodiments of the instant disclosure, the sizes of the beads of the instant disclosure tend to range from 1 μm to 100 μm in diameter (with all subranges within this range expressly contemplated), e.g., depending upon the extent of image resolution desired, nature of the solid support to be used for spatial bead array construction, sequencing processes (e.g., flow cell sequencing) to be employed, as well as other factors. In some embodiments, the 1 μm to 100 μm diameter beads include porous polystyrene, porous polymethacrylate and/or polyacrylamide.

In some embodiments, the beads are 1 μm to 40 μm in diameter. In some embodiments, the beads are about 10 μm in diameter. In some embodiments, arrays in which beads are used include, without limitation, those having beads in wells, beads arranged upon a flat surface (e.g., a slide), optionally beads captured upon a flat surface (e.g., a layer of beads adhered to or otherwise stably associated with a slide (e.g., a layer of beads adsorbed to a slide-attached elastomeric surface).

In some embodiments, a capture material is used to associate a bead with a spot of the substrate. In some embodiments, the capture material is a liquid electrical tape. An exemplary liquid electrical tape of the instant disclosure is Permatex™ liquid electrical tape, which is a weatherproof protectant for wiring and electrical connections. Liquid capture material such as liquid tape can be applied as a liquid, which then dries to a vinyl polymer that resists dirt, dust, chemicals, and moisture, ensuring that applied beads are attached to a capture material-coated slide in a dry condition. Without wishing to be bound by theory, it is believed that one advantage of the instant methods is that the oligonucleotide-coated beads used in certain embodiments of the invention, which are attached to a solid support (e.g., a slide surface via use, e.g., of electrical tape as a capture material) are maintained in a dry state that optimizes transfer of DNA from a section (e.g., a cryosection) of a tissue to a bead-coated surface (again without wishing to be bound by theory, such transfer is currently believed to occur via capillary action at the scale of the microbead-tissue section interface surface). It is believed that this highly efficient and direct transfer of cellular DNA (i.e., the whole or partial genome, mtDNA, viral and/or bacterial DNA, etc. of cells found within sectioned tissues) to microbeads (where each microbead respectively possesses thousands of oligonucleotides capable of capturing oligoribonucleotides, e.g., transcripts) arrayed upon a solid support—where the transfer occurs upon an otherwise dry surface, therefore limiting and/or eliminating diffusive properties—is one feature that imparts the instant methods and compositions with extremely high resolution (i.e., resolution at 10-50 μm spacing across a two-dimensional image of a section) of assessment of the cellular DNA molecules of assayed tissue sections.

In certain aspects of the disclosure, beads are immobilized to a spot of the substrate surface, and the location of spots is known or determined prior to use of the substrate surface in the assay system. In another aspect, the beads are immobilized onto separate structural elements that are then provided in known locations on the substrate surface. In yet another aspect, the beads may be provided in or on features of the substrate surface, e.g., provided in wells or channels. In some embodiments, the beads are arranged in an organized pattern in the array. In some embodiments, the beads are randomly distributed in the array.

An array can be placed within a capture area of a substrate, and a substrate can include multiple capture areas, each comprising an array. As used herein, the “capture area” or “measurement area” is a discrete area on the substrate surface where an array is located. These capture areas can be formed by spatially selective deposition of the spots on the substrate surface. In some embodiments, the arrays are arranged on the substrate into segments of one or more capture areas for reagent distribution and agent determination. These regions may be physically separated using barriers or channels. They may still comprise several additional discrete measurement areas with agents that are different or in different combination from each other. In some embodiments, the substrate comprises at least two capture areas. In some embodiments, the substrate comprises at least 3 capture areas. In some embodiments, the substrate comprises at least 4 capture areas. In some embodiments, the substrate comprises at least 5 capture areas In some embodiments, the substrate comprises at least 6 capture areas. In some embodiments, the substrate comprises at least 7 capture areas. In some embodiments, the substrate comprises at least 8 capture areas. In some embodiments, the substrate comprises at least 9 capture areas. In some embodiments, the substrate comprises at least 10 capture areas. The multiple capture areas can be arranged on the substrate in any possible

In some embodiments, a capture area has a dimension of about 1 mm² to about 100 mm². In some embodiments, the capture area has a dimension of 100 mm² or less. In some embodiments, the capture area has a dimension of 75 mm² or less. In some embodiments, the capture area has a dimension of 50 mm² or less. In some embodiments, the capture area has a dimension of 25 mm² or less. In some embodiments, the capture area has a dimension of 15 mm² or less. In some embodiments, the capture area has a dimension of 10 mm² or less. In some embodiments, the capture area has a dimension of about 100 mm². In some embodiments, the capture area has a dimension of about 75 mm². In some embodiments, the capture area has a dimension of about 50 mm². In some embodiments, the capture area has a dimension of about 25 mm². In some embodiments, the capture area has a dimension of about 15 mm². In some embodiments, the capture area has a dimension of about 14 mm². In some embodiments, the capture area has a dimension of about 13 mm². In some embodiments, the capture area has a dimension of about 12 mm². In some embodiments, the capture area has a dimension of about 11 mm². In some embodiments, the capture area has a dimension of about 10 mm². In some embodiments, the capture area has a dimension of about 9 mm². In some embodiments, the capture area has a dimension of about 8 mm². In some embodiments, the capture area has a dimension of about 7 mm². In some embodiments, the capture area has a dimension of about 6 mm². In some embodiments, the capture area has a dimension of about 5 mm². In some embodiments, the capture area has a dimension of about 4 mm². In some embodiments, the capture area has a dimension of about 3 mm². In some embodiments, the capture area has a dimension of about 2 mm². In some embodiments, the capture area has a dimension of about 1 mm². In some embodiments, the capture area has a dimension of up to 10 mm². In some embodiments, the capture area has a dimension of up to 6.5 mm². In some embodiments, the capture area has a dimension of up to 3 mm².

In some embodiments, the array spots are about 20 μm to about 125 μm apart as measured from the center of spot to the center of an adjacent spot. In some embodiments, the spots are less than 125 μm apart as measured from the center of spot to the center of an adjacent spot. In some embodiments, the spots are less than 100 μm apart as measured from the center of spot to the center of an adjacent spot. In some embodiments, the spots are less than 75 μm apart as measured from the center of spot to the center of an adjacent spot. In some embodiments, the spots are less than 50 μm apart as measured from the center of spot to the center of an adjacent spot. In some embodiments, the spots are less than 25 μm apart as measured from the center of spot to the center of an adjacent spot. In some embodiments, the spots are less than 10 μm apart as measured from the center of spot to the center of an adjacent spot. In some embodiments, the spots are less than 5 μm apart as measured from the center of spot to the center of an adjacent spot. In some embodiments, the spots are less than 1 μm apart as measured from the center of spot to the center of an adjacent spot. In some embodiments, the spots are less than 900 nm apart as measured from the center of spot to the center of an adjacent spot. In some embodiments, the spots are less than 750 nm apart as measured from the center of spot to the center of an adjacent spot. In some embodiments, the spots are less than 500 nm apart as measured from the center of spot to the center of an adjacent spot.

In embodiments of the disclosure, the size and layout of the spots are combined in any combination of the size of the spots and the distance of the spots as measured from center of spot to center of spot described herein. For example, in some embodiments, the spots are less than about 60 μm in diameter and are no more than about 100 μm apart as measured by center of spot to center of spot. In some embodiments, the spots are less than about 40 μm in diameter and are no more than about 50 μm apart as measured by center of spot to center of spot. In some embodiments, the spots are less than about 220 nm in diameter and are no more than about 750 nm apart as measured by center of spot to center of spot.

As used herein, “spatial resolution” refers to the measure of the smallest object that can be resolved by the array represented by each pixel. The spatial resolution in the present context is determined by the size of the pixel. In some embodiments, the spatial resolution is determined by the size of the spot. In some embodiments, the spatial resolution is determined by the size of the bead. In some embodiments, the spatial resolution ranges from about 10 nm to about 1 mm. In some embodiments, the spatial resolution is about 100 nm to about 500 microns. In some embodiments, the spatial resolution is about 1 micron to about 250 microns. In some embodiments, the spatial resolution ranges from about 5 microns to about 100 microns. In some embodiments, the spatial resolution ranges from about 10 microns to about 75 microns. In some embodiments, the spatial resolution is about 75 microns. In some embodiments, the spatial resolution is about 70 microns. In some embodiments, the spatial resolution is about 65 microns. In some embodiments, the spatial resolution is about 60 microns. In some embodiments, the spatial resolution is about 55 microns. In some embodiments, the spatial resolution is about 50 microns. In some embodiments, the spatial resolution is about 25 microns. In some embodiments, the spatial resolution is about 10 microns. In some embodiments, the spatial resolution is less than 10 microns and at a subcellular level. In some embodiments, the spatial resolution is about 5 microns. In some embodiments, the spatial resolution is about 1 micron.

Embodiments of the disclosure comprise DNA oligomers in the spots immobilized on the substrate. In some embodiments, the spots comprise nucleic acids immobilized directly or indirectly to the substrate surface, e.g., directly through the use of amino groups on the substrate surface or indirectly through the use of a linker. The location of the nucleic acid sequences is known or determined prior to use of the substrate surface in the assay system. In some embodiments, the nucleic acids may be immobilized directly or indirectly onto beads that are then provided in known locations on the substrate surface. In some embodiments, the nucleic acids may be provided in or on features of the substrate surface, e.g., provided in wells.

Numerous methods can be used for the deposition of the spots and the DNA oligomers of the spots. For example, the DNA oligomers can be delivered together or separately from the spot. If delivered together they can be attached (e.g., synthesized as a single molecule or attached through ligation or a chemical coupling mechanism) or simply mixed together to be attached after delivery to the substrate. In a preferred aspect, the spot and the oligomer are made separately, mixed together for attachment, and delivered either attached or as a mixture to be attached on the substrate. In a specific aspect the spots are delivered generally over the substrate surface and the oligomers are delivered in a pattern-specific manner.

Examples of methods that can be used for deposition of spots onto the substrate surface include, but are not limited to, ink jet spotting, mechanical spotting by means of pin, pen or capillary, micro contact printing, fluidically contacting the measurement areas with the biological or biochemical or synthetic recognition elements upon their supply in parallel or crossed micro channels, upon exposure to pressure differences or to electric or electromagnetic potentials, and photochemical or photolithographic immobilization methods.

The spots or beads can be deposited on the substrate in a pre-determined pattern or in a random arrangement. For example, the assay system can utilize an encoding scheme that comprises a 2-dimensional grid format based on the discrete positioning of the binding agents in the substrate surfaces. In another example, the spatial patterns may be based on more randomized cell locations, e.g., the patterns on the substrate surface follow an underlying biological structure rather than a strict, x,y grid pattern. As used herein, the term “random” can be used to refer to the spatial arrangement or composition of locations on a surface. For example, there are at least two types of order for an array described herein, the first relating to the spacing and relative location of features (also called “sites”) and the second relating to identity or predetermined knowledge of the particular species of molecule that is present at a particular feature. Accordingly, features of an array can be randomly spaced such that nearest neighbor features have variable spacing between each other. Alternatively, the spacing between features can be ordered, for example, forming a regular pattern such as a rectilinear grid or hexagonal grid. In another respect, features of an array can be random with respect to the identity or predetermined knowledge of the species of analyte (e.g., nucleic acid of a particular sequence) that occupies each feature independent of whether spacing produces a random pattern or ordered pattern. An array set forth herein can be ordered in one respect and random in another. For example, in some embodiments set forth herein a surface is contacted with a population of nucleic acids under conditions where the nucleic acids attach at sites that are ordered with respect to their relative locations but ‘randomly located’ with respect to knowledge of the sequence for the nucleic acid species present at any particular site. Reference to “randomly distributing” nucleic acids at locations on a surface is intended to refer to the absence of knowledge or absence of predetermination regarding which nucleic acid will be captured at which location (regardless of whether the locations are arranged in an ordered pattern or not).

Accordingly, the instant methods can employ an array of beads, wherein different nucleic acid probes are attached to different beads in the array. In such embodiments, each bead can be attached to a different nucleic acid probe and the beads can be randomly distributed on the substrate in order to effectively attach the different nucleic acid probes to the substrate. Optionally, the substrate can include wells having dimensions that accommodate no more than a single bead. In such a configuration, the beads may be attached to the wells due to forces resulting from the fit of the beads in the wells. As described elsewhere herein, it is also possible to use attachment chemistries or capture materials (e.g., liquid electrical tape) to adhere or otherwise stably associate the beads with a substrate, optionally including holding the beads in wells that may or may not be present on a substrate.

Nucleic acid probes that are attached to beads can include barcode sequences. A population of the beads can be configured such that each bead is attached to only one type of barcode (e.g., a spatial barcode) and many different beads each with a different barcode are present in the population. In this embodiment, randomly distributing the beads to a substrate will result in randomly locating the nucleic acid probe-presenting beads (and their respective barcode sequences) on the substrate. In some cases, there can be multiple beads with the same barcode sequence such that there is redundancy in the population. However, randomly distributing a redundancy-comprising population of beads on a substrate—especially one that has a capacity that is greater than the number of unique barcodes in the bead population—will tend to result in redundancy of barcodes on the substrate, which will tend to reduce image resolution in the context of the instant disclosure (i.e., where the precise location of a barcoded bead cannot be resolved due to redundancy of barcode use within an arrayed population of beads, it is contemplated that such redundant locations will simply be eliminated from an ultimate image produced by methods of the instant disclosure, or other modes of adjustment (e.g., normalization and/or averaging of values) may also be employed to address such redundancies). Alternatively, in preferred embodiments, the number of different barcodes in a population of beads can exceed the capacity of the substrate in order to produce an array that is not redundant with respect to the population of barcodes on the substrate. The capacity of the substrate will be determined in some embodiments by the number of features (e.g. single bead occupancy wells) that attach or otherwise accommodate a bead.

In certain embodiments, each DNA oligomer comprises a spatial barcode and a poly deoxythymine (dT) sequence. The DNA oligomers comprise a spatial barcode wherein all DNA oligomers in one spot share the same spatial barcode, which is different from spatial barcodes of other spots of the array. As used herein, the term “spatial barcode” is intended to mean a nucleic acid having a sequence that is indicative of a location. Typically, the nucleic acid is a synthetic molecule having a sequence that is not found in one or more biological specimen that will be used with the nucleic acid. However, in some embodiments the nucleic acid molecule can be naturally derived, or the sequence of the nucleic acid can be naturally occurring, for example, in a biological specimen that is used with the nucleic acid. The location indicated by a spatial barcode can be a location in or on a biological specimen, in or on a substrate or a combination thereof. In some embodiments, the identification of the barcode is determined after a population of spots (each possessing a distinct barcode sequence) has been arrayed upon a substrate (optionally randomly arrayed upon a substrate) and sequencing of such a spot-associated barcode sequence has been determined in situ upon the substrate.

In some embodiments, the assay utilizes two or more oligonucleotides, the oligonucleotides comprising a universal primer region and a region that correlates specifically to a single spatial pattern within the spatial encoding scheme. In a specific embodiment, the assay comprises two allele specific oligonucleotides and one locus specific oligonucleotides. These oligonucleotides allow the identification of specific SNPs, indels or mutations within an allele. This is useful in the identification of genetic changes in somatic cells, genotyping of tissues, and the like.

In some embodiments, the DNA oligomers comprises an oligonucleotide sequence. In some embodiments, the DNA oligomers comprise a unique molecular identifier (UMI) sequence. The UMIs are complex indices added to sequencing libraries before any PCR amplification steps, enabling the accurate bioinformatic identification of PCR duplicates. UMIs are also known in the art as “Molecular Barcodes” or “Random Barcodes” and consist of short random nucleotide sequences which are added to each molecule in a sample as a unique identifier tag. In some embodiments, the DNA oligomers comprise an oligonucleotide sequence and a UMI sequence. In some embodiments, step (a) also includes DNA oligomers that act as primers.

In some embodiments, the poly(dT) sequence is an oligo d(T) VN. Oligo d(T) VN is used for the priming and sequencing of mRNA adjacent to the 3′-poly A tail. An oligo d(T) VN is a poly(dT) that comprises a VN sequence at the 3′ end wherein the V represents either A, C, or G nucleotides while N is any nucleotide base (i.e. A, C, G, or T). Including VN at 3′ end of RT primer enriches for non-poly A region of transcripts whereas without the VN, RT can be primed in the poly A region so cDNA would have A/T homopolymer which is undesirable in some sequencing applications.

### The Sample

The methods disclosed herein are advantageous in that they are compatible with numerous sample types, such as such as fresh samples, such as primary tissue sections, and preserved samples including but not limited to frozen samples and paraformalin-fixed, paraffin-embedded (FFPE) samples. In some embodiments, the biological sample is a tissue. As used herein, the term “tissue” is intended to mean an aggregation of cells, and, optionally, intercellular matter. Typically, the cells in a tissue are not free floating in solution and instead are attached to each other to form a multicellular structure. Exemplary tissue types include muscle, nerve, epidermal and connective tissues. In some embodiments, the muscle tissue is a cardiac, skeletal, or smooth muscle tissue. In some embodiments, the epithelial tissue is simple squamous, stratified squamous, simple cuboidal, stratified cuboidal, simple columnar, stratified columnar, pseudostratified columnar, or transitional epithelia. In some embodiments, the connective tissue is connective tissue proper or specialized connective tissue. In some embodiments, the connective tissue proper is loose or dense tissue, comprising collagen, reticular, or elastic fibers. In some embodiments, the specialized connective tissue comprises adipose, cartilage, bone, blood, reticular, and lymphatic tissues. In some embodiments, the biological sample is a combination of tissue types which form an organ. For example, the biological sample comprises at least two types of tissue selected from connective tissue, epithelial tissue, muscle tissue, and nervous tissue. In some embodiments, the biological sample is taken from a testis.

In some embodiments, the biological sample the biological sample is a tissue sample of an injured tissue or an organ suspected to suffer an infection. In some embodiments, the tissue sample is a tumor section, gut microbiome, brain section, patient biopsy, or a plant sample. In some embodiments, the biological sample may be from a human, mammal other than human, invertebrate, plant, fungi, bacteria, virus, archaea, or other living species. In some embodiments, the biological sample is a tissue sample of an injured tissue, or an organ suspected to suffer an infection. In some embodiments, the tissue sample is a tumor section, gut microbiome, brain section, patient biopsy, or a plant sample.

In some embodiments, step (b) further comprises fixing the biological sample (e.g., using formaldehyde, Formalin-fixed, parafin-embedded (FFPE), Acetone, Methanol and acetone, Glyoxal fixation, and methacarn fixation). A tissue can be prepared in any convenient or desired way for its use in a method, composition or apparatus herein. Fresh, frozen, fixed or unfixed tissues can be used. A tissue can be fixed or embedded using methods described herein or known in the art.

A tissue sample for use herein, can be fixed by deep freezing at temperature suitable to maintain or preserve the integrity of the tissue structure, e.g. less than −20° C. In another example, a tissue can be prepared using formalin-fixation and paraffin embedding (FFPE) methods which are known in the art. Other fixatives and/or embedding materials can be used as desired. A fixed or embedded tissue sample can be sectioned, i.e. thinly sliced, using known methods. For example, a tissue sample can be sectioned using a chilled microtome or cryostat, set at a temperature suitable to maintain both the structural integrity of the tissue sample and the chemical properties of the nucleic acids in the sample. Exemplary additional fixatives that are expressly contemplated include alcohol fixation (e.g., methanol fixation, ethanol fixation), glutaraldehyde fixation and paraformaldehyde fixation.

In some embodiments, a tissue sample will be treated to remove embedding material (e.g. to remove paraffin or formalin) from the sample prior to release, capture or modification of nucleic acids. This can be achieved by contacting the sample with an appropriate solvent (e.g. xylene and ethanol washes). Treatment can occur prior to contacting the tissue sample with a solid support-captured bead array as set forth herein or the treatment can occur while the tissue sample is on the solid support-captured bead array.

Exemplary methods for manipulating tissues for use with solid supports to which nucleic acids are attached are set forth in US Pat. App. Publ. No. 2014/0066318, which is incorporated herein by reference.

In some embodiments, step (b) further comprises staining the fixed biological sample. Such staining is done through histology staining and immunostaining procedures known in the art. Such staining procedures include Hematoxylin and Eosin (H&E), immunostaining, Azan Rapid Stain, Congo Red Staining, Cresyl Fast Violet, Giemsa Staining, luxol fast blue, Masson's Trichrome staining; Mallory's Muscle Fiber Stain, Nissl Staining, Thionin Nissl staining, and toluidine blue staining. In some embodiments, a tissue is permeabilized and the cells of the tissue lysed. Any of a variety of art-recognized lysis treatments can be used. Target nucleic acids that are released from a tissue that is permeabilized can be captured by nucleic acid probes, as described herein and as known in the art. For example, permeabilization can occur by incubating the sample with 0.5% Triton-X for 30 min. For whole genome preparation, two methods can be used to permeabilize the tissue and deplete nucleosomes: 1) Tissue is treated with 0.5% Triton-X for 30 min, washed, and incubated with 0.1N HCl for 5 min. 2) Tissue is treated with SDS (range from 0.8%-8%, diluted into water) for 10 min at 60° C., followed by 1.5% Triton-X (diluted in water) for 10 minutes, washed, and optionally incubated with proteinase K (range from 1-20 ug/mL) for 10 minutes at 37° C.

In some embodiments, step (b) comprises capturing and/or recording an image of the fixed and stained biological sample.

The method disclosed herein are used to detect, quantify. and spatially determine all types of RNA. In some embodiments, the RNA is non-coding RNA. In some embodiments, the RNA is coding RNA. In some embodiments, the RNA detected are ribonucleic acids (RNAs), RNA degradation products, RNAs comprising a poly(A) tail, messenger RNA (mRNA), long noncoding RNAs (lncRNAs), long intergenic noncoding RNAs (lincRNAs), cis-natural antisense transcripts (cisNATs), antisense RNAs, ribosomal RNAs (rRNAs), microRNAs (miRNAs), small interfering RNAs (siRNAs), short hairpin RNAs (shRNAs), guide RNAs (gRNAs), transfer RNAs (tRNAs), small nuclear RNAs (snRNAs), small nucleolar RNAs (snoRNAs), small Cajal body-specific RNA (scaRNAs), enhancer RNAs (eRNAs), piwi-interacting RNAs (piRNAs), Y RNAs, non-coding RNAs, vault RNA, viral RNA, microbial RNA such as bacterial RNA, archaeal RNA, or fungal RNA, or combinations thereof. In some embodiments, the RNA spatially detected are viral RNA, bacterial RNA, archaeal RNA, fungal RNA, or a combination thereof.

### Polyadenylation

Step (c) of the disclosed methods includes contacting the substrate comprising the biological sample with a Poly(A) polymerase enzyme mix. The Poly(A) polymerase (also called polynucleotide adenylyltransferases or PAP) enzyme of step (c) catalyzes the incorporation of adenine residues into the 3′ termini of RNA, effectively adding a poly(A) tail to RNA. Poly(A) polymerases suitable for use in this disclosure are specific to polyadenylate only RNA molecules. Poly(A) polymerase adds poly(A) tails to RNA where the RNA molecules do not already have a poly(A) tail and will also enhance or lengthen the poly(A) tails on RNA molecules that already have poly(A) tails. In some embodiments, the Poly(A) polymerase enzyme is a yeast Poly(A) polymerase (such as Thermo Scientific, cat #74225Z25KU), an E. coli Poly(A) polymerase, or any other Poly(A) polymerase that is specific to polyadenylate only RNA molecules. In some embodiments, the Poly(A) enzyme mix comprises Poly(A) polymerase (polynucleotide adenylytransferase) and a polymerase reaction buffer reagent.

In some embodiments, step (c) further comprises, prior to the contacting step, equilibrating the substrate by adding a wash buffer to the substrate. In some embodiments, this wash buffer comprises Poly(A) polymerase reaction buffer, an RNase inhibitor, and nuclease free water. In some embodiments, step (c) comprises after the equilibrating, adding a Poly(A) polymerase enzyme mix which comprises Poly(A) polymerase reaction buffer, a Poly(A) polymerase enzyme, adenosine triphosphate (ATP), RNase inhibitor, and nuclease-free water and incubating.

In some embodiments, the length of the poly(A) tail is controlled in the in situ polyadenylation. In such embodiments, the poly(A) tail length is controlled through the incorporation of (i) ATP and (ii) biotin-11-ATP or dATP. In some embodiments, the Poly(A) polymerase enzyme mix includes (i) ATP and (ii) biotin-11-ATP or dATP at a ratio of at least 5:1. In some embodiments, the Poly(A) polymerase enzyme mix includes (i) ATP and (ii) biotin-11-ATP or dATP at a ratio of at least 4:1. In some embodiments, the Poly(A) polymerase enzyme mix includes (i) ATP and (ii) biotin-11-ATP or dATP at a ratio of at least 3:1. In some embodiments, the Poly(A) polymerase enzyme mix includes (i) ATP and (ii) biotin-11-ATP or dATP at a ratio of at least 2:1. In some embodiments, the Poly(A) polymerase enzyme mix includes (i) ATP and (ii) biotin-11-ATP or dATP at a 1:1 ratio.

In some embodiments, the poly(A) tail is at least about 10 base pairs in length. In some embodiments, the poly(A) tail is at least about 100 base pairs in length. In some embodiments, the poly(A) tail is at least about 250 base pairs in length. In some embodiments, the poly(A) tail is at least about 300 base pairs in length. In some embodiments, the poly(A) tail is at least about 400 base pairs in length. In some embodiments, the poly(A) tail is at least about 500 base pairs in length. In some embodiments, the poly(A) tail is at least about 750 base pairs in length. In some embodiments, the poly(A) tail is at least about 1000 base pairs in length. In some embodiments, the length of the poly(A) tail is less than about 2,000 base pairs. In some embodiments, the length of the poly(A) tail is less than about 1,600 base pairs. In some embodiments, the length of the poly(A) tail is less than about 1,000 base pairs. In some embodiments, the length of the poly(A) tail is about 10 base pairs to about 4,000 base pairs. In some embodiments, the length of the poly(A) tail about 100 base pairs to about 3,000 base pairs. In some embodiments, the length of the poly(A) tail is about 500 base pairs to about 2,000 base pairs. In some embodiments, the length of the poly(A) tail is about 800 base pairs to about 1,600 base pairs.

In some embodiments, step (d) includes permeabilizing the biological sample. A permeabilization step allows for the release of RNA from the biological sample, and hence, allows for capture of the RNA molecules from the biological sample. Certain embodiments of the instant disclosure feature permeabilizing agents, examples of which tend to compromise and/or remove the protective boundary of lipids often surrounding cellular macromolecules. Disruption of cellular lipid barriers via administration of a permeabilizing agent can provide enhanced physical access to cellular macromolecules, such as DNA, that might otherwise be relatively inaccessible. Specifically contemplated examples of permeabilizing agents include, without limitation: Triton X-100, NP-40, methanol, acetone, Tween 20, saponin, Leucoperm™, and digitonin, among others.

Some embodiments of the current disclosure comprise (e) adding reverse transcription reagents to generate cDNA molecules from captured RNA molecules, wherein cDNA molecules generated from RNAs captured by DNA oligomers on a spot comprise a spatial barcode common to the spot. In some embodiments, step (e) further comprises initiating second strand synthesis via the addition of a second strand primer.

In some embodiments, the cDNAs generated are denatured and transferred from the spots for amplification. Methods of the instant disclosure can employ any of a variety of amplification techniques. Exemplary amplification techniques that can be used include, but are not limited to, polymerase chain reaction (PCR), rolling circle amplification (RCA), multiple displacement amplification (MDA), and random prime amplification (RPA). In some embodiments the amplification can be carried out in solution, for example, when features of an array are capable of containing amplicons in a volume having a desired capacity. In certain embodiments, an amplification technique used in a method of the present disclosure will be carried out on solid phase. For example, one or more primer species (e.g. universal primers for one or more universal primer binding site present in a nucleic acid probe) can be attached to a bead or other solid support. In PCR embodiments, one or both of the primers used for amplification can be attached to a bead or other solid support (e.g. via a gel). Formats that utilize two species of primers attached to a bead or other solid support are often referred to as bridge amplification because double stranded amplicons form a bridge-like structure between the two surface-attached primers that flank the template sequence that has been copied. Exemplary reagents and conditions that can be used for bridge amplification are described, for example, in U.S. Pat. Nos. 5,641,658; 7,115,400; and 8,895,249; and/or U.S. Patent Publication Nos. 2002/0055100 A1, 2004/0096853 A1, 2004/0002090 A1, 2007/0128624 A1 and 2008/0009420 A1, each of which is incorporated herein by reference. Solid-phase PCR amplification can also be carried out with one of the amplification primers attached to a bead or other solid support and the second primer in solution. An exemplary format that uses a combination of a surface-attached primer and soluble primer is the format used in emulsion PCR as described, for example, in Dressman et al., Proc. Natl. Acad. Sci. USA 100:8817-8822 (2003), WO 05/010145, or U.S. Patent Publication Nos. 2005/0130173 or 2005/0064460, each of which is incorporated herein by reference. Emulsion PCR is illustrative of the format, and it will be understood that for purposes of the methods set forth herein the use of an emulsion is optional and indeed for several embodiments an emulsion is not used.

In some embodiments, the amplified cDNAs are further processed to reach optimal amplicon size by way of methods commonly known in the art. Briefly, full length, PCR-amplified cDNA molecules are enzymatically fragmented and then further amplified with an additional round of PCR. The fragmentation is performed for an amount of time, which yields the desired size distribution where a longer fragmentation results in shorter amplicons. If the amplicon length is too short, the PCR product size will be too similar to the primer dimer to distinguish them from one another without sequencing. If it is too long, the PCR efficiency will decrease requiring more time for elongation and a great probability of non-specific amplification. As used herein, the term “amplicon,” when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template including, for example, polymerase extension, polymerase chain reaction (PCR), rolling circle amplification (RCA), multiple displacement amplification (MDA), ligation extension, or ligation chain reaction. An amplicon can be a nucleic acid molecule having a single copy of a particular nucleotide sequence (e.g. a PCR product) or multiple copies of the nucleotide sequence (e.g. a concatameric product of RCA). A first amplicon of a target nucleic acid is typically a complementary copy. Subsequent amplicons are copies that are created, after generation of the first amplicon, from the target nucleic acid or from the first amplicon. A subsequent amplicon can have a sequence that is substantially complementary to the target nucleic acid or substantially identical to the target nucleic acid. In some embodiments, the optimal amplicon size depends on many variables and the design preferences. In some embodiments, the optimal amplicon size ranges between 20 to 1,500 base pairs. In some embodiments, the optimal amplicon size differs for quantitative PCR and standard PCR. In some embodiments, the optimal amplicon size for quantitative PCR ranges between 20 to 1,000 base pairs. In some embodiments, the optimal amplicon size ranges between 200 and 1,500 base pairs for standard PCR.

In some embodiments of the current disclosure, the sequences of the generated cDNA are obtained. In some embodiments, the generated cDNAs with spatial barcodes and the sequences of the generated cDNAs are used to map the spatial gene expression. In some embodiments, the generated cDNAs and the sequences of the generated cDNA are correlated with the captured image of the fixed and stained biological sample in order to map the spatial gene expression. One embodiment of the disclosed method includes a step of correlating locations in an image of a biological specimen with barcode sequences of nucleic acid probes that are attached to individual spots to which the biological specimen is, was, or will be contacted. Accordingly, characteristics of the biological specimen that are identifiable in the image can be correlated with the nucleic acids that are found to be present in their proximity. Any of a variety of morphological characteristics can be used in such a correlation, including for example, cell shape, cell size, tissue shape, staining patterns, presence of particular proteins (e.g. as detected by immunohistochemical stains) or other characteristics that are routinely evaluated in pathology or research applications. Accordingly, the biological state of a tissue or its components as determined by visual observation can be correlated with molecular biological characteristics as determined by spatially resolved nucleic acid analysis.

In some embodiments, the method further comprises comparing the spatial gene expression map of the tissue sample to (i) the spatial gene expression map of a control sample, or (ii) the spatial gene expression map of another sample of the same tissue taken at a different time point. In such embodiments, the spatial gene expression map of a control sample may be that of healthy tissue while the spatial gene expression map of the tissue sample is suspected of infection, disease, or injury. In some embodiments, the tissue sample is thought to have a genetic defect and is compared to a control sample without the suspected defect.

In some embodiments, the method further comprises isolating a subpopulation of cDNAs from the cDNAs generated in step (e). In some embodiments, this isolation of a subpopulation of cDNAs occurs through co-immunoprecipitation or bio pull-down assays. Such assays allow for the generated cDNAs to be incubated with specific oligo probes. The specific oligo probes can be directed to a desired set of cDNA to give a more specific result of the desired RNA. For example, the oligo probes can be directed to viral RNA and the generated cDNA to viral RNA can be isolated. In some embodiments, the isolated subpopulation of selected cDNAs is generated from viral RNAs, bacterial RNA, archaeal RNA, or fungal RNA and therefore, viral RNA, bacterial RNA, archaeal RNA, or fungal RNA are isolated. In some embodiments, the sequences of the cDNAs in the isolated subpopulation are obtained.

In some embodiments, the method of spatially detecting any type of RNA can be used with any platform that recognizes polyadenylated RNA molecules. In certain aspects involving nucleic acid agents, any methods of sequence determination can be used, e.g., sequencing, hybridization and the like. In a preferred aspect, nucleic acid sequencing, and preferably next-generation sequencing, is used to decode the spatial encoding scheme in the assay system of the invention. This provides a very wide dynamic range for very large numbers of assays, allowing for efficient multiplexing. Many of these platforms are known in the art and are commercially available. Examples of such commercially available platforms are Visium Spatial Gene Expression assay from 10× Genomics, GeoMx Digital Spatial Profiler from NanoString, and HCR RNA-FISH Technology from Molecular Instruments.

### Kit

Another aspect of the current disclosure is directed to kits containing agents of this disclosure for use in the methods of the present disclosure. Kits of the instant disclosure may include one or more containers comprising an agent. In such aspects, the kit is for use in spatially determining RNA molecules, wherein the kit comprises (1) a substrate defined by an array of spots or beads, wherein each spot or bead comprises DNA oligomers (for capturing and priming of polyadenylated RNAs) immobilized on the substrate, and wherein each of the DNA oligomers comprises: (i) a spatial barcode, wherein all primers in one spot share the same spatial barcode, which is different from the spatial barcodes in other spots; and (ii) a poly(dT) sequence; and (2) at least one reagent comprising a Poly(A) polymerase enzyme mix.

In some embodiments, the substrate of the kit is a solid, planar, and/or rigid substrate or support which is insoluble in aqueous liquid. The substrate can be non-porous or porous. The substrate can optionally be capable of taking up a liquid (e.g. due to porosity) but will typically be sufficiently rigid that the substrate does not swell substantially when taking up the liquid and does not contract substantially when the liquid is removed by drying. A nonporous solid support is generally impermeable to liquids or gases. In some embodiments, the substrate of the kit is a solid support composed of a material selected from the group consisting of glass, silicon, poly-L-lysine coated material, nitrocellulose, polystyrene, cyclic olefin copolymers (COCs), cyclic olefin polymers (COPs), polypropylene, polyethylene and polycarbonate.

In some embodiments, the substrate comprises at least one array, where each array comprises a plurality of spots. In some embodiments, the spots of the various supplied arrays range from about 10 nm to about 1 mm in diameter. Generally, the spots within one array are of substantially the same size. In some embodiments, the spots are about 100 nm to 1 mm in diameter. In some embodiments, the spots are about 150 nm to about 70 μm in diameter. In some embodiments, the spots are between about 200 nm and about 65 μm in diameter. In some embodiments, the spots are between about 220 nm and about 60 μm in diameter. In some embodiments, the spots are about 10 μm to about 40 μm in diameter. In some embodiments, the spots are about 50 μm to about 70 μm in diameter.

In some embodiments, the substrate of the kit comprises at least one array that is arranged in a capture area. These capture areas can be formed by spatially selective deposition of the spots on the substrate surface. In some embodiments, the arrays are arranged on the substrate into segments of one or more capture areas for reagent distribution and agent determination. These regions may be physically separated using barriers or channels. They may still comprise several additional discrete measurement areas with agents that are different or in different combination from each other. In some embodiments, a capture area has a dimension of about 1 mm² to about 100 mm².

In some embodiments, the array of spots comprises a range of 10 spots to 100,000,000 spots. In some embodiments, the array comprises any range of spots between 10 and any number up to and including 100,000,000 spots. In some embodiments, the array comprises 10 to 10,000,000 spots. In some embodiments, the array comprises 10 to 1,000,000 spots. In some embodiments, the array comprises 10 to 500,000 spots. In some embodiments, the array comprises 10 to 250,000 spots. In some embodiments, the array comprises 10 to 100,000 spots. In some embodiments, the array comprises 10 to 50,000 spots. In some embodiments, the array comprises 10 to 1,000 spots.

In some embodiments, the DNA oligomers are immobilized to the substrate directly. In some embodiments, the DNA oligomers are immobilized to the substrate indirectly. In embodiments where the DNA oligomers are immobilized to the substrate indirectly, such indirect immobilization can occur through beads or other particles to which the oligomers attach. In some embodiments, one bead is present for each spot within an array. In some embodiments, multiple beads are present at each spot within an array.

In some embodiments of the disclosure, the size and layout of the spots are combined in any combination of the spot size and the distance of the spots as measured from center of spot to center of spot. For example, in some embodiments, the spots are less than 60 μm in diameter and are no more than 100 μm apart as measured by center of spot to center of spot. In some embodiments, the spots are less than about 40 μm in diameter and are no more than about 50 μm apart as measured by center of spot to center of spot. In some embodiments, the spots are less than 220 nm in diameter and are no more than 750 nm apart as measured by center of spot to center of spot.

In some embodiments of the disclosure, the size and layout of the beads are combined in any combination of the bead size and the distance of the beads as measured from center of bead to center of bead. For example, in some embodiments, the beads are less than 60 μm in diameter and are no more than 100 μm apart as measured by center of bead to center of bead. In some embodiments, the spots are less than about 40 μm in diameter and are no more than about 50 μm apart as measured by center of spot to center of spot. In some embodiments, the beads are less than 220 nm in diameter and are no more than 750 nm apart as measured by center of bead to center of bead.

In some embodiments, the spatial resolution of the provided array in the disclosed kit ranges from about from about 10 nm to about 1 mm. In some embodiments, the spatial resolution of the provided array in the disclosed kit ranges from about 1 micron to about 100 microns, or about 5 microns to about 75 microns.

In some embodiments, instructions for use are included in the kit. In some embodiments, the user is directed to a website for instructions.

In some embodiments, the DNA oligomers further comprises an oligonucleotide sequence; and/or a unique molecular identifier (UMI) sequence as described above.

In some embodiments, the Poly(A) polymerase enzyme mix comprises (1) a polymerase reaction buffer reagent; (2) a poly(A) polymerase enzyme reagent; and (3) optionally a nuclease free water reagent. In some embodiments, the Poly(A) polymerase enzyme mix further comprises adenosine triphosphate reagent and/or an RNase inhibitor reagent. (1) a polymerase reaction buffer reagent; (2) a poly(A) polymerase enzyme reagent; (3) ATP; (4) biotin-11-ATP or dATP, and (5) optionally a nuclease free water reagent. In some embodiments, the ATP to biotin-11-ATP or dATP is at a ratio that greater than about 5:1.

In some embodiments, the kit comprises a poly(dT) sequence that is an oligo d(T) VN. An oligo d(T) VN is a poly(dT) that comprises a VN sequence at the 3′ end wherein the V represents either A, C, or G nucleotides while N is any nucleotide base (i.e. A, C, G, or T). Including VN at 3′ end of RT primer enriches for non-poly A region of transcripts whereas without the VN, RT can be primed in the poly A region so cDNA would have A/T homopolymer which is undesirable in some sequencing applications.

In some embodiments, the kit further comprises a wash buffer reagent. In some embodiments, the wash buffer reagent comprises (1) a polymerase reaction buffer reagent; (2) an RNase inhibitor reagent; and optionally (3) a nuclease-free water reagent.

In some embodiments, the reagents included in the kit are either ready to use, concentrated, lyophilized, or a combination of ready to use and concentrated. In some embodiments, the reagents included in the kit are provided in separate containers or provided in pre-mixed quantities of any combination of reagents.

### EXAMPLES

The steps of the method described in the various examples disclosed herein are sufficient to carry out the methods of the present disclosure. Thus, in an example, a method consists essentially of a combination of the steps of the methods disclosed herein. In another example, a method consists of such steps.

The following examples are presented to illustrate the present disclosure. The examples are not intended to be limiting in any manner.

### Example 1. Spatial Mapping of mRNA Using the Visium Protocol

As explained above, the disclosed method of spatially mapping the total transcriptome is compatible with any available protocol involving spatial mapping of polyadenylated RNA molecules. The disclosed method allows for the total transcriptome to be spatially mapped and in this example, the protocol for the Visium Spatial Gene Expression Solution available from 10× Genomics was used. The following protocol is from the Visium Spatial Gene Expression Reagent Kits User Guide supplied on the 10× Genomics website and with its kits for spatially mapping mRNA of a tissue sample.

The Visium Spatial Gene Expression Solution is said to measure total mRNA in intact tissue sections and maps the location(s) where gene activity is occurring. Each Visium Spatial Gene Expression Slide contains Capture Areas with gene expression spots that include primers required for capture and priming of poly-adenylated mRNA. Tissue sections placed on these Capture Areas are permeabilized and cellular mRNA is captured by the primers on the gene expression spots. All the cDNA generated from mRNA captured by primers on a specific spot share a common Spatial Barcode. Libraries are generated from the cDNA and sequenced and the Spatial Barcodes are used to associate the reads back to the tissue section images for spatial gene expression mapping.

This example outlines the protocol for generating Visium Spatial Single Cell 3′ Gene Expression libraries from tissue sections placed on the Capture Areas of a Visium Spatial Gene Expression Slide.

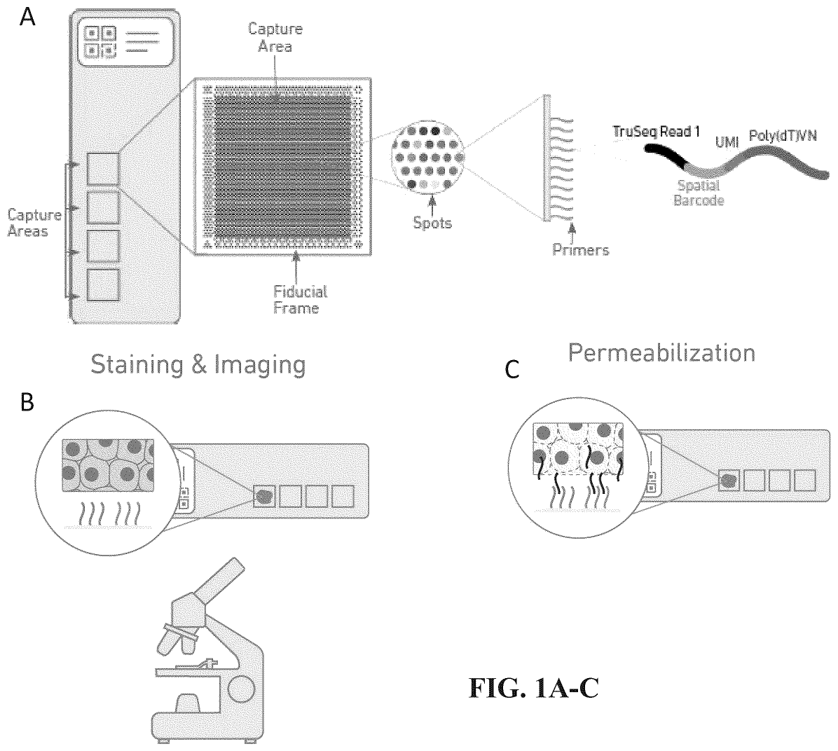

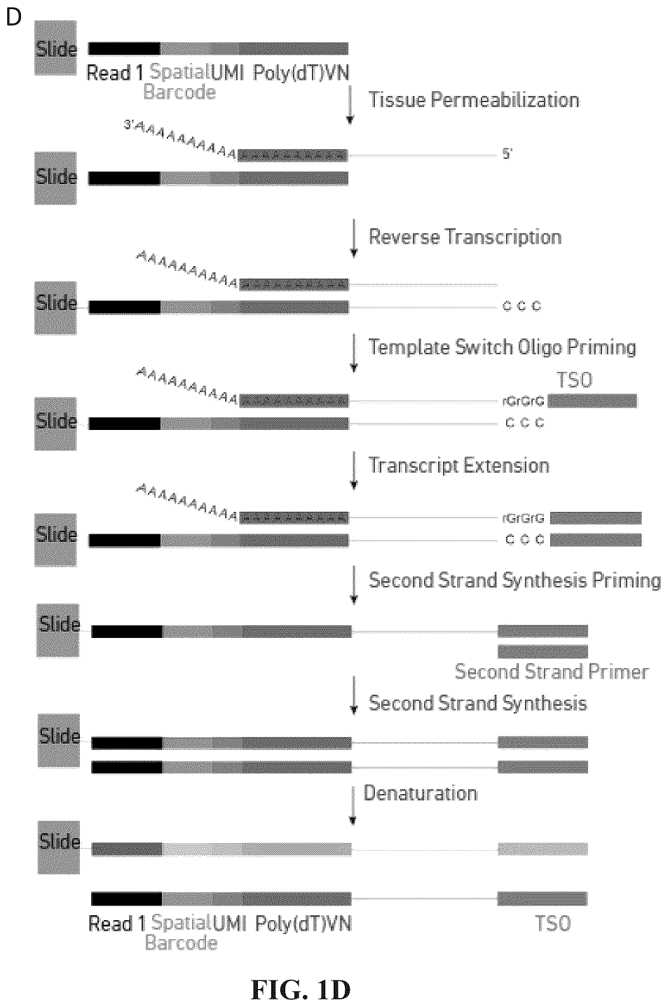

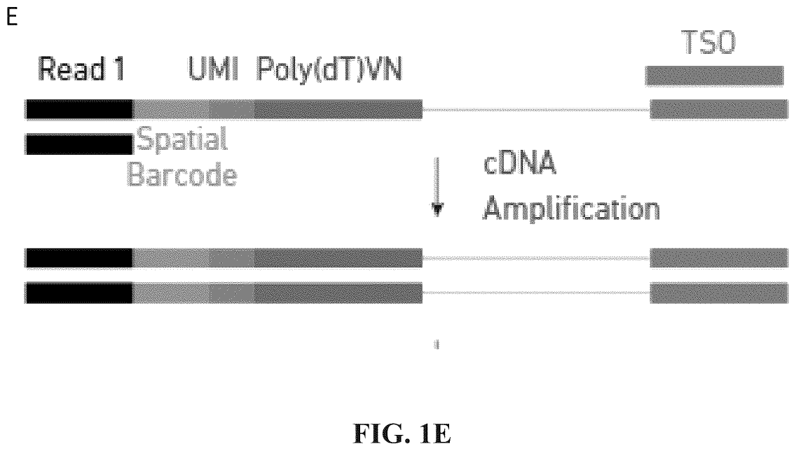

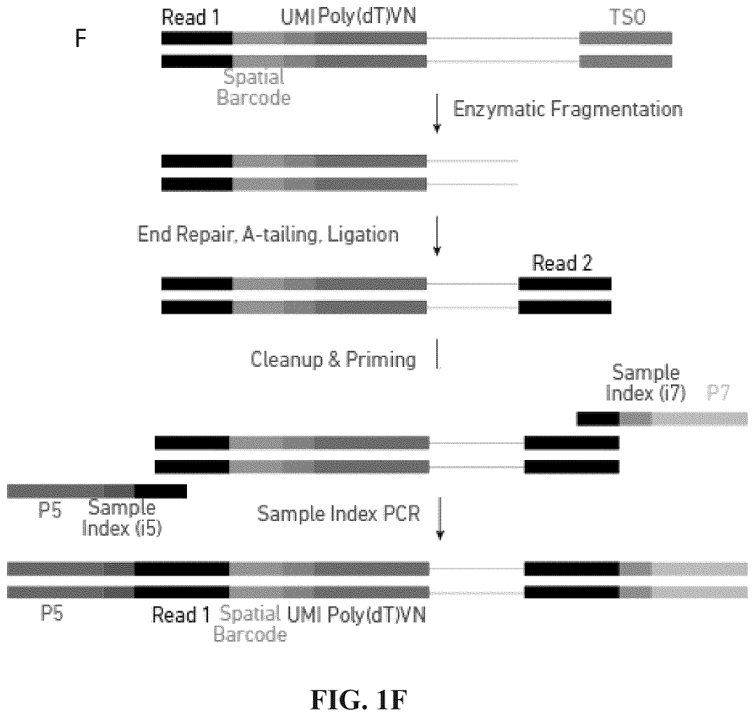

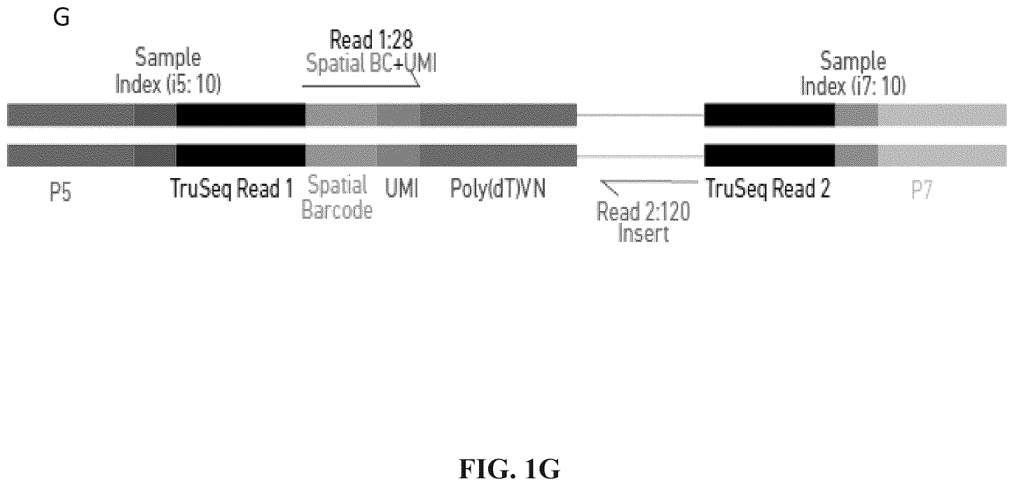

The Visium Spatial Gene Expression Slide includes 4 Capture Areas (6.5×6.5 mm), each defined by a fiducial frame (fiducial frame +Capture Area is 8×8 mm) (FIG. 1A). The Capture Area has ˜5,000 gene expression spots, each spot with primers that include:
Illumina TruSeq Read 1 (partial read 1 sequencing primer)
16 nt Spatial Barcode (all primers in a specific spot share the same Spatial Barcode)
12 nt unique molecular identifier (UMI)
30 nt poly(dT) sequence (captures poly-adenylated mRNA for cDNA synthesis).

### Step 1 of Visium Protocol: Tissue Staining and Imaging (FIG. 1B)

Tissue sections on the Capture Areas of the Visium Spatial Gene Expression were fixed using methanol. Hematoxylin was used to stain the nuclei, followed by eosin staining for the extracellular matrix and cytoplasm. The stained tissue sections were imaged. The images were used downstream to map the gene expression patterns back to the tissue sections.

### Step 2: Permeabilization & Reverse Transcription (FIG. 1C)

A Permeabilization Enzyme was used to permeabilize the tissue sections on the slide. The poly-adenylated mRNA released from the overlying cells was captured by the primers on the spots. RT Master Mix containing reverse transcription reagents was added to the permeabilized tissue sections. Incubation with the reagents produced spatially barcoded, full-length cDNA from poly-adenylated mRNA on the slide.

### Step 3: Second Strand Synthesis and Denaturation (FIG. 1D)

Second Strand Mix was added to the tissue sections on the slide to initiate second strand synthesis. This was followed by denaturation and transfer of the cDNA from each Capture Area to a corresponding tube for amplification and library construction.

### Step 4: cDNA Amplification and Quality Control (FIG. 1E)

After transfer of cDNA from the slide, spatially barcoded, full-length cDNA was amplified via PCR to generate sufficient mass for library construction.

### Step 5: Visium Spatial Gene Expression Library Construction (FIG. 1F)

Enzymatic fragmentation and size selection were used to optimize the cDNA amplicon size. P5, P7, i7 and i5 sample indexes, and TruSeq Read 2 (read 2 primer sequence) were added via End Repair, A-tailing, Adaptor Ligation, and PCR. The final libraries contain the P5 and P7 primers used in Illumina amplification.

### Step 6: Sequencing (FIG. 1G)

A Visium Spatial Gene Expression library comprises standard Illumina paired-end constructs which begin and end with P5 and P7. The 16 bp Spatial Barcode and 12 bp UMI were encoded in Read 1, while Read 2 was used to sequence the cDNA fragment. i7 and i5 sample index sequences are incorporated. TruSeq Read 1 and TruSeq Read 2 are standard Illumina sequencing primer sites used in paired-end sequencing.

### Example 2. In Situ Poly(A) Tailing in Mouse Gut Tissue

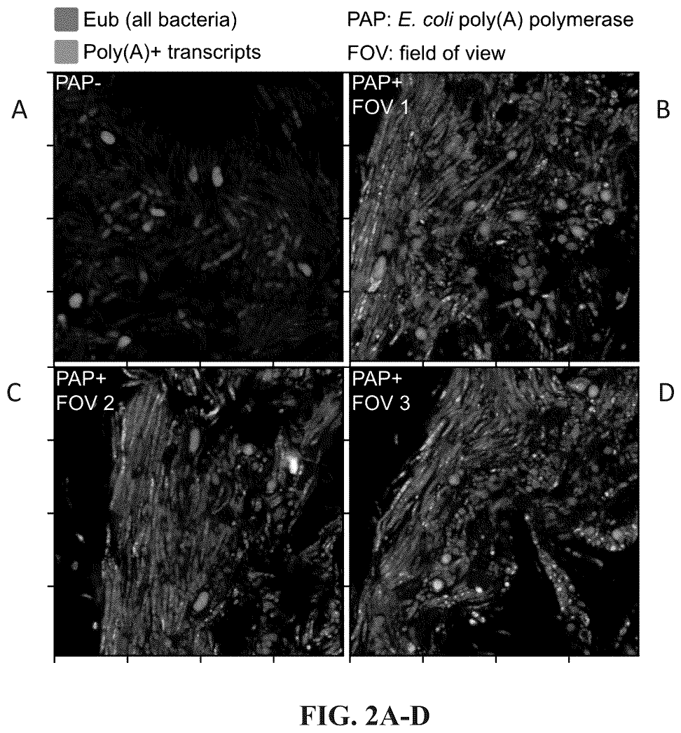

To show proof of concept for in situ poly(A) tailing was possible, mouse gut tissue was harvested and fixed. Then the tissue was subjected to E. coli poly(A) polymerase in order to add poly(A) tails. The E. coli poly(A) polymerase was able to polyadenylate the tissue as seen in FIG. 2. The upper left panel is negative control, where no poly(A) polymerase was used. The other panels show multiple fields of views where microbial transcripts are poly(A) tailed by E. coli poly(A) polymerase and detected by poly(T) fluorescent probes.

### Example 3. In Situ Polyadenylation Enables Capture of Coding and Noncoding RNAs

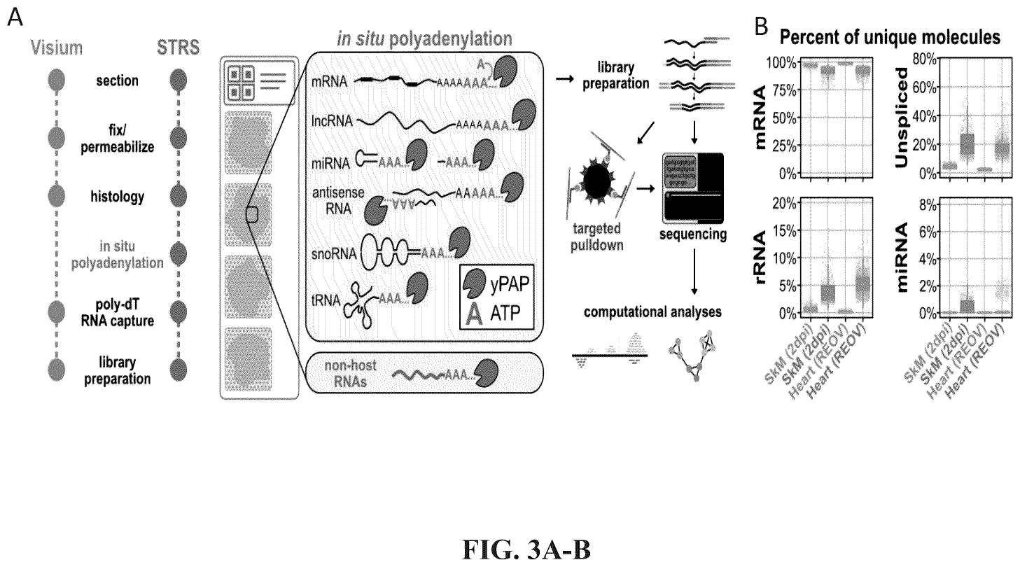

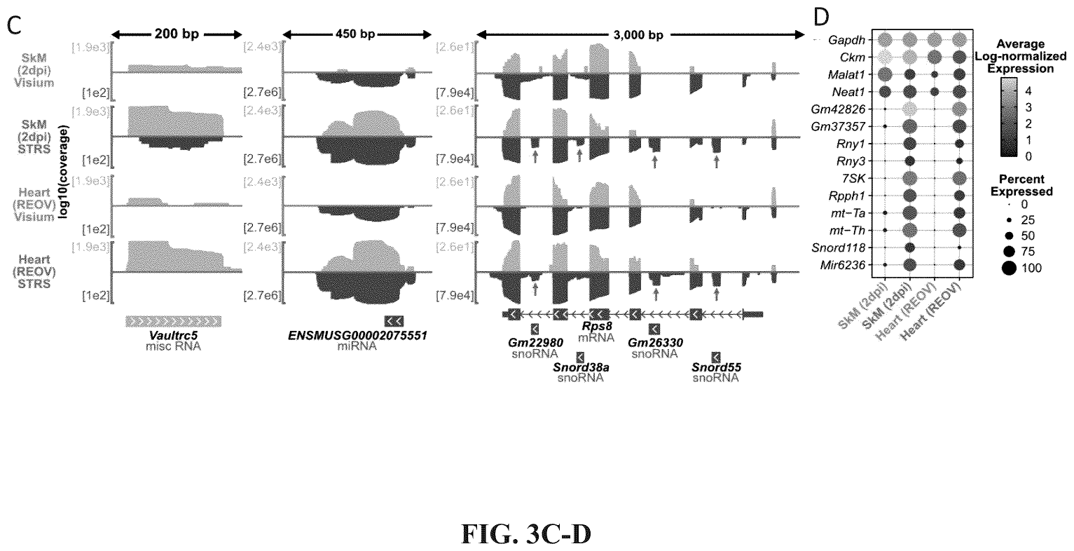

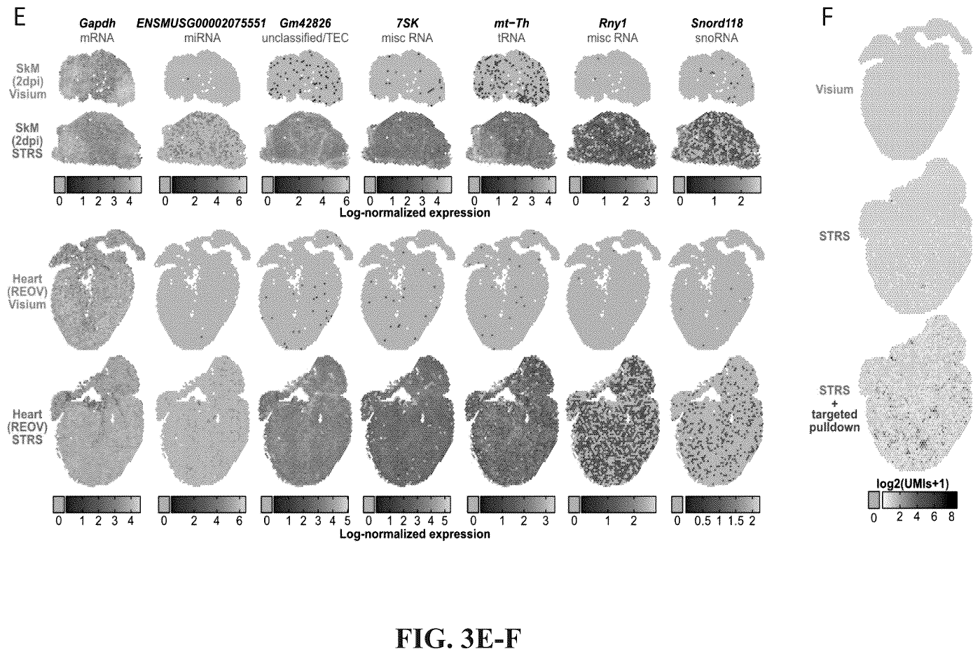

Spatial Total RNA-Sequencing (STRS) deviates a commercially available method for spatial RNA-sequencing to capture the total transcriptome, not just mRNA as described in Example 1. The biological sample was first sectioned, fixed with methanol, and stained for histology. After imaging, the sample was rehydrated and then incubated with yeast poly(A) polymerase for 25 minutes at 37° C. The yeast poly(A) polymerase adds poly(A) tails to the 3′ end of all RNAs so that endogenous poly(A) tails are extended and non-A-tailed transcripts are polyadenylated. After in situ polyadenylation, STRS again follows the commercially available protocol without modification (FIG. 3A). One feature of the Visium method leveraged in STRS, is its use of a strand-aware library preparation. It was found that strandedness is desirable for the study of noncoding and antisense RNAs (see below)

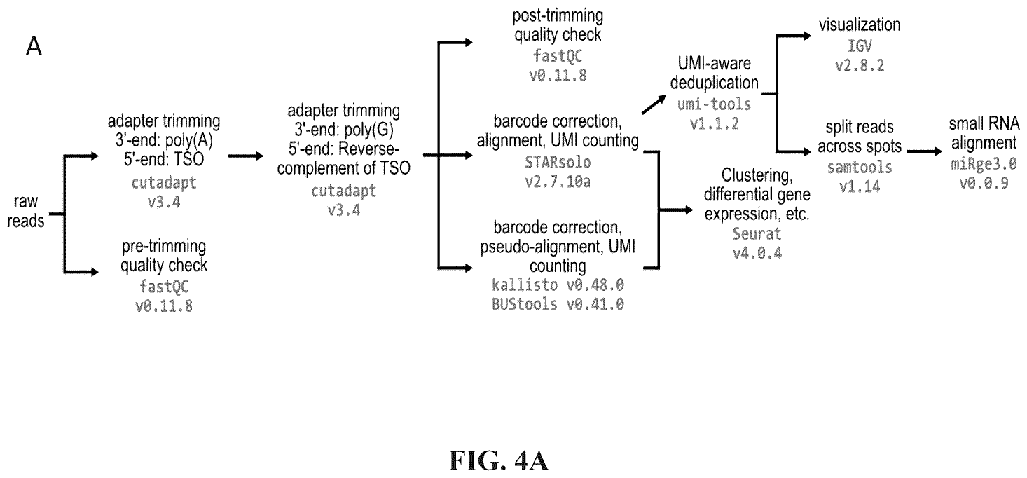

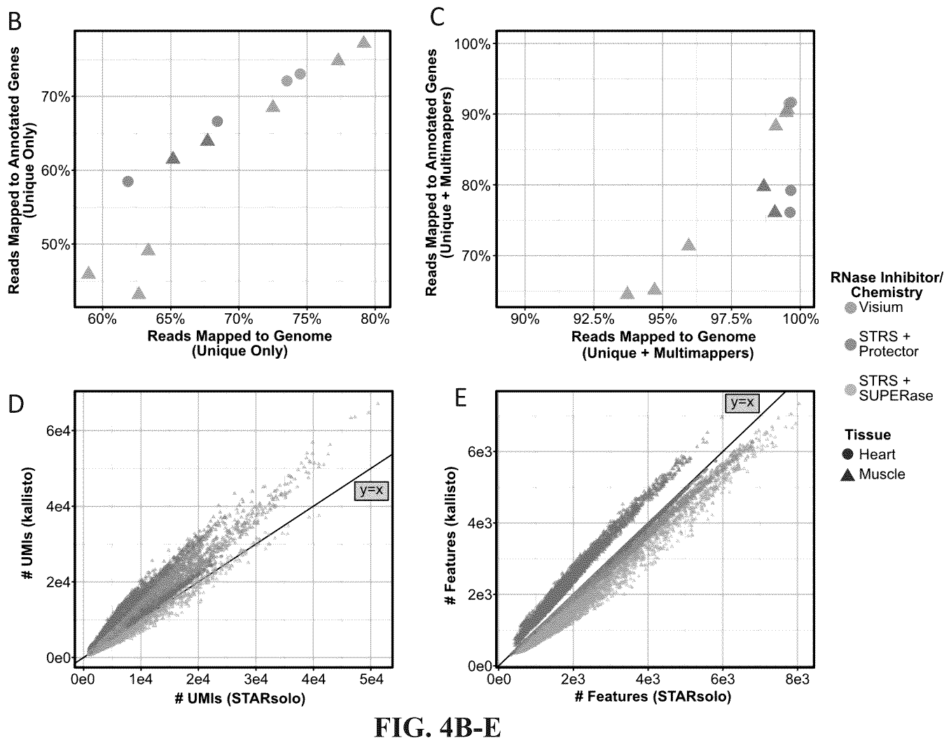

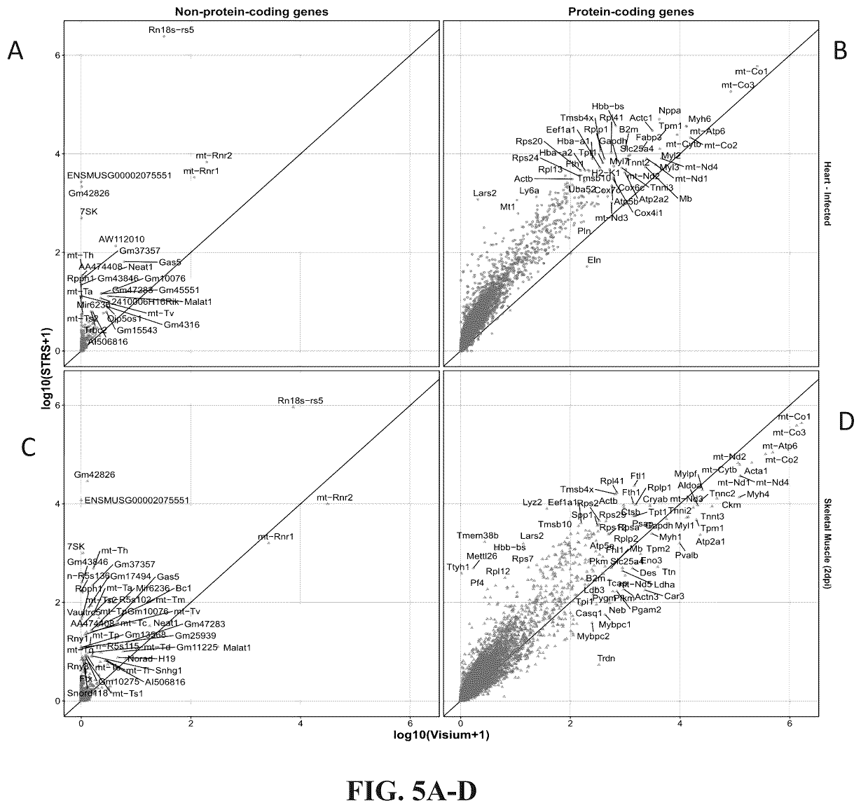

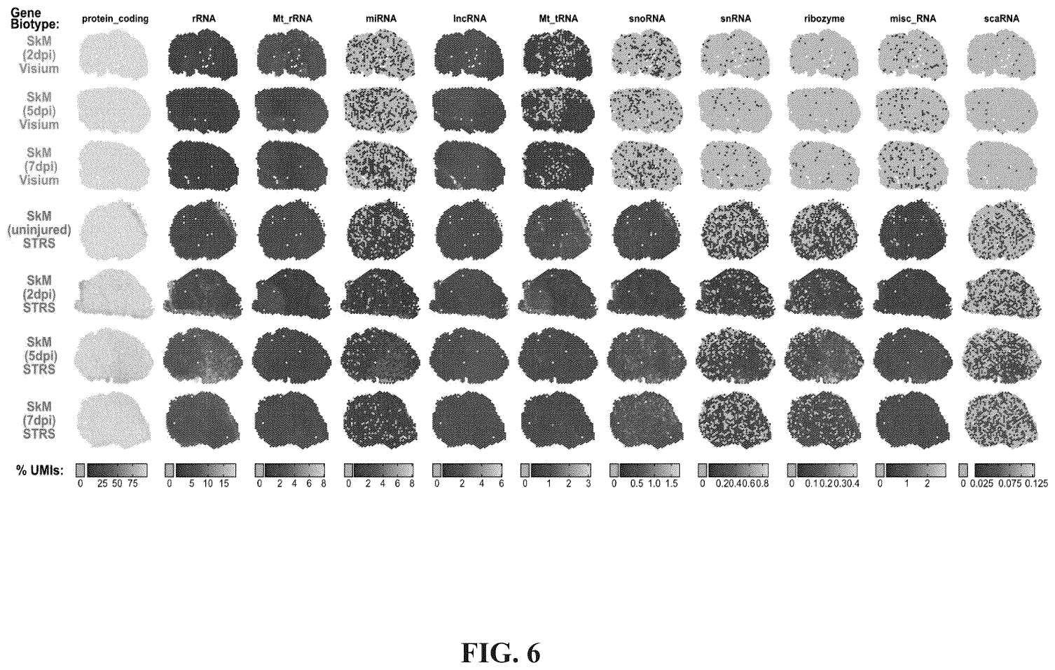

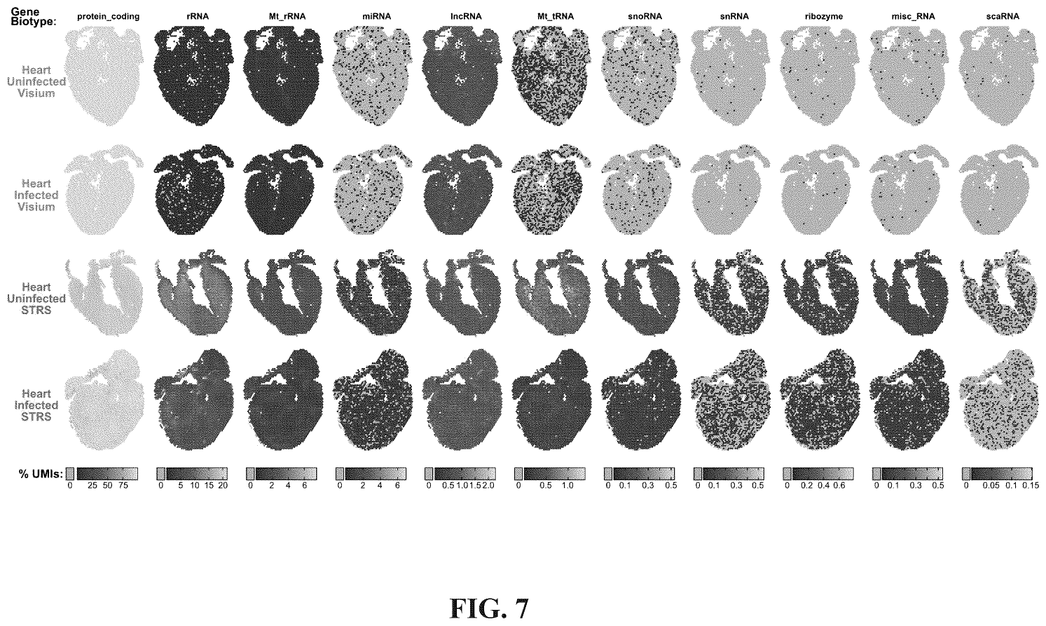

In order to test the performance and versatility of STRS, the disclosed method was applied to two distinct mouse tissue types: injured hindlimb muscle and virally infected heart tissue. The percentage of unique molecules (UMIs) was quantified as a function of RNA biotype (GENCODE M28 annotations; FIG. 3B). The Visium method was used as a control. Compared to the control, similar counts for protein coding and other endogenously polyadenylated transcripts were found (FIG. 4 and FIG. 5). STRS enabled robust detection of several types of noncoding RNAs which are poorly recovered or not detected at all by the Visium method, including ribosomal RNAs (rRNAs; mean of 5.4% and 2.6% of UMIs for STRS and Visium respectively), microRNAs (miRNAs; 0.4% in STRS versus 0.004% in Visium), transfer RNAs (tRNAs; 0.4% in STRS versus 0.02% in Visium), small nucleolar RNAs (snoRNAs; 0.2% in STRS versus 0.002% in Visium), and several other biotypes (FIG. 3B, FIG. 6, FIG. 7). STRS libraries also had an increased fraction of unspliced transcripts (2.7% in Visium versus 18.3% in STRS). Unspliced or nascent RNA counts have previously been used to predict transcriptional trajectories for single cells. Improved detection of nascent RNAs may enable more accurate trajectory imputation and reveal the dynamics of spatial gene expression. Finally, STRS libraries had an increased fraction of reads which map to intergenic regions, reflecting an increased capture of unannotated transcriptional products (22.2% in STRS versus 9.5% in Visium; FIGS. 4B and 4C). STRS captured many RNAs which were not present in Visium libraries. Many of these features map outside of or antisense to known annotations (FIG. 3C). STRS also detected many noncoding transcripts which are intragenic to other genes (FIG. 3C). Standard short-read sequencing was sufficient to delineate these features from the surrounding host genes, as reflected by the expression count matrices for STRS versus the Visium data (FIG. 3D). Importantly, the STRS method spatially mapped each of these features and visualized spatial patterns of gene expression (FIG. 3E). It was found that features which were incompletely annotated (ENSMUSG00002075551) showed sparse spatial expression. Several highly abundant genes showed homogenous patterns of expression, reflecting putative (Gm42826) or known (7SK) housekeeping roles.

Next, the ability of in situ polyadenylation to enable capture of non-A-tailed viral RNA was tested. To this end, murine heart tissues infected with Type 1-Lang reovirus (REOV) was assayed. REOV is a segmented double-stranded RNA virus that expresses ten transcripts which are not polyadenylated. No reovirus transcripts were detected with the Visium workflow, whereas STRS enabled detection of more than 200 UMIs representing all ten reovirus gene segments (FIG. 3F). To deeply profile viral RNAs, targeted enrichment of viral-derived cDNA from the final sequencing libraries and re-sequenced the products was performed. This enrichment led to a further ˜26-fold increase of the mean viral UMIs captured per spot (minimum L1 segment with 262 UMIs, maximum S4 segment with 1095 UMIs). Taken together, these findings demonstrate that STRS enables the study of many types of RNAs that are not detectable with existing technologies.

### Example 4. Spatial Total RNA-Sequencing Reveals Spatial Patterns of Gene Regulation in Skeletal Muscle Regeneration

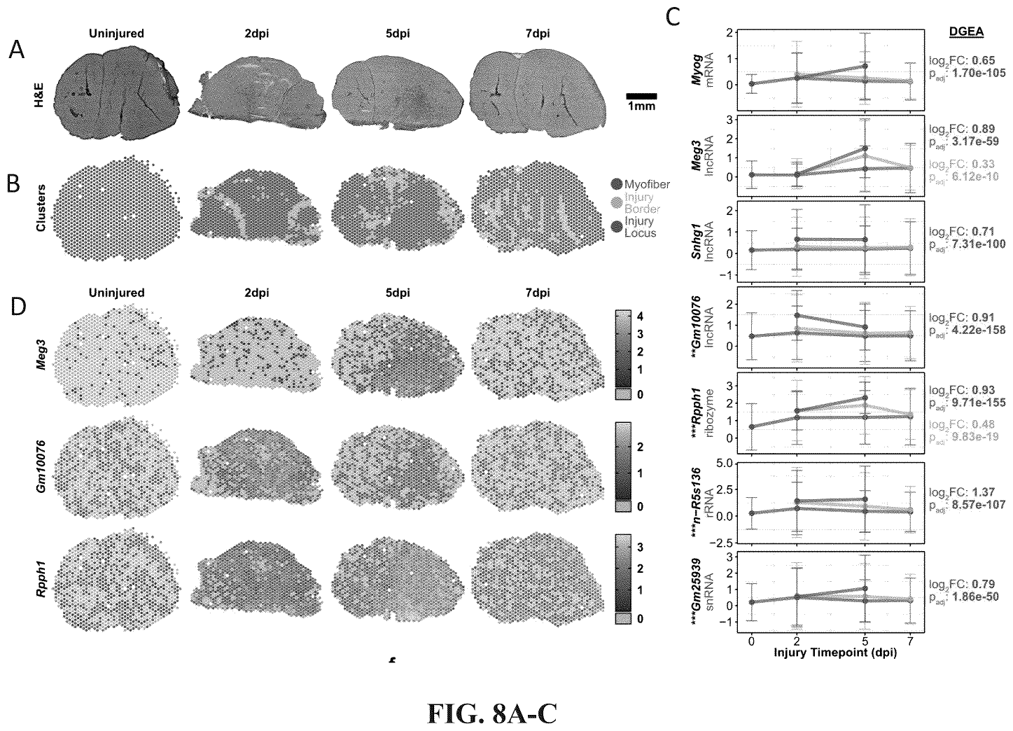

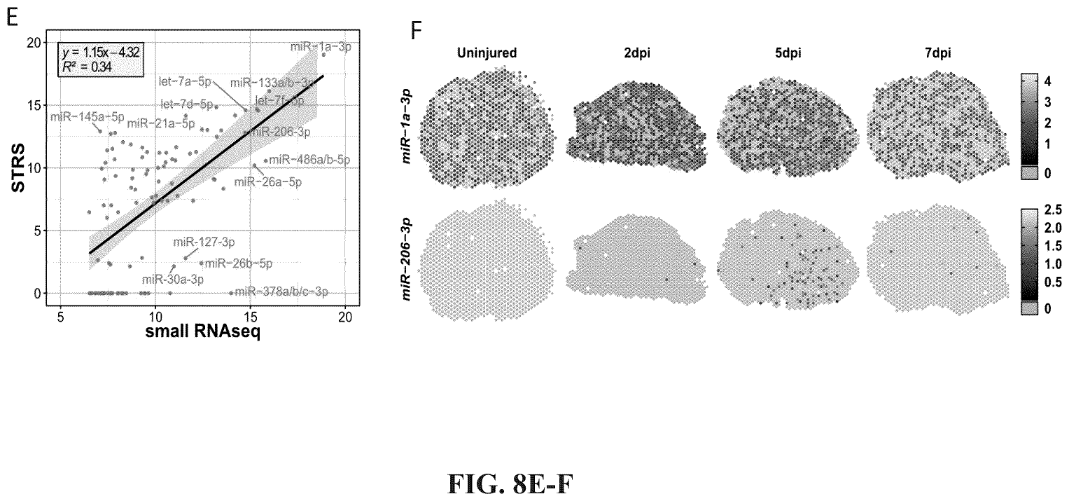

Skeletal muscle regeneration is a coordinated system guided by complex gene regulatory networks. STRS was applied to spatially map the coding and noncoding transcriptome in a mouse model of skeletal muscle regeneration. To induce muscle injury, both tibialis anterior muscles of old (20 months) C57BL/6J mice were injected with 10 μl of notexin (10 μg/ml; Latoxan; France). Either before injury or 2-, 5-, or 7-days post-injury (dpi), mice were sacrificed, and tibialis anterior muscles collected. After dissection, samples were embedded in O.C.T. Compound (Tissue-Tek) and frozen fresh in liquid nitrogen. H&E imaging showed immune infiltration in the middle of tissue sections at 2 and 5 dpi, which was mostly resolved by 7 dpi (FIG. 8A). Unsupervised clustering identified spots in the injury loci, spots around the border of the injury loci, and spots under intact myofibers (FIG. 8B). Spot UMI counts as generated by kallisto were used. First, counts were log-normalized and scaled using default parameters with Seurat. Principal component analysis was then performed on the top 2000 most variable features for each tissue slice individually. Finally, unsupervised clustering was performed using the ‘FindClusters( )’ function from Seurat. The top principal components which accounted for 95% of variance within the data were used for clustering. For skeletal muscle samples, a clustering resolution was set to 0.8. For heart samples, clustering resolution was set to 1.0. Default options were used for all other parameters. Finally, clusters were merged according to similar gene expression patterns and based on histology of the tissue under each subcluster.

Differential gene expression analysis across the regional clusters was performed to identify noncoding RNAs specific to the injury locus (FIG. 8C). Differential gene expression analysis was performed using the ‘FindAllMarkers( )’ function from Seurat. Default parameters were used, including the use of the Wilcoxon ranked sum test to identify differentially expressed genes. To identify features enriched in the skeletal muscle STRS datasets, all Visium and STRS were first merged and compared according to the method used (Visium vs. STRS). To identify cluster-specific gene expression patterns, skeletal muscle samples were first clustered as described above individually. STRS samples were then merged, and differential gene expression analysis was performed across the three injury region groups.

Several RNAs were found which were spatiotemporally associated with injury locus, many of which are undetected or poorly detected by Visium (FIGS. 8C and 8D). Meg3 is an endogenously polyadenylated lncRNA which has been shown to regulate myoblast differentiation in vitro. Meg3 expression was confined to the injury locus at 5 dpi, when myoblast differentiation and myocyte fusion occurs. Gm10076, a transcript with a biotype annotation conflict (Ensembl: lncRNA; NCBI: pseudogene) and no known function, was highly and specifically expressed within the injury locus 2 dpi. Gm10076 expression was reduced but still localized to the injury site by 5 dpi and returned to baseline levels by 7 dpi. Rpph1, a ribozyme and component of the RNase P ribonucleoprotein which has also been shown to play roles in tRNA and lncRNA biogenesis, showed broad expression by 2 dpi which peaked and localized to the injury site at 5 dpi. It was also found that STRS captured high levels of antisense transcripts for Rpph1 which were not detected by the Visium chemistry. This demonstrated that STRS can robustly profile both polyadenylated and non-polyadenylated RNAs across heterogeneous tissues.

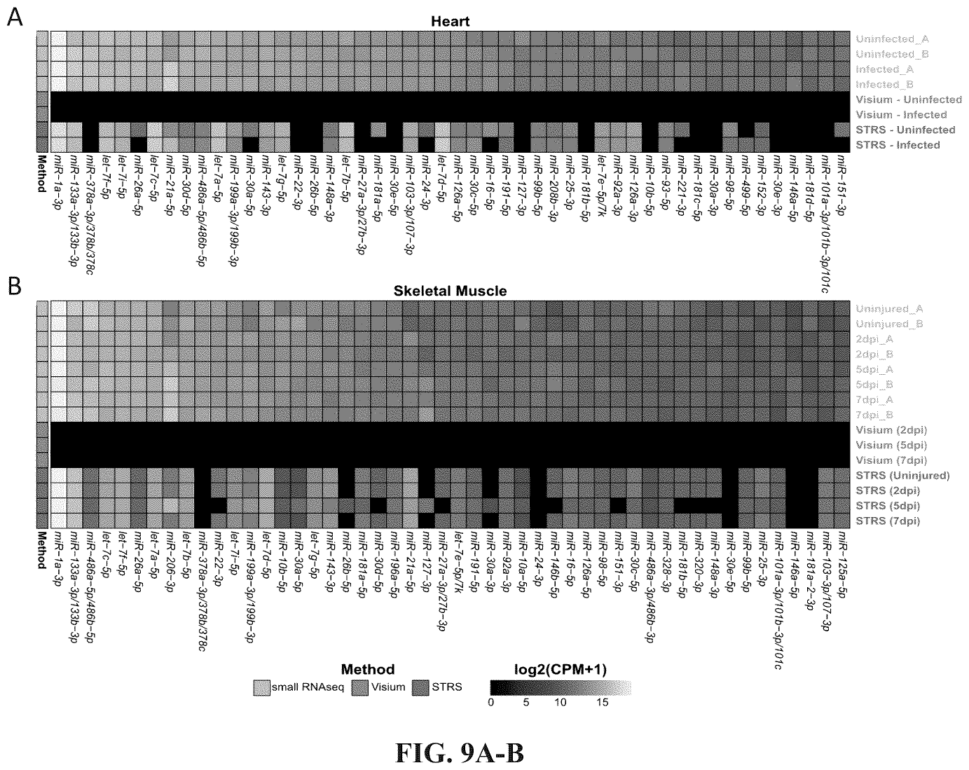

The role of miRNAs in skeletal muscle regeneration is well-established in the art. Mature miRNAs are about 22 nucleotides long, not polyadenylated, and not captured by the standard Visium workflow (FIG. 9). Determination if STRS was able to detect mature miRNAs was necessary. Matched bulk small RNAseq libraries were first generated from entire tibialis anterior muscles as a gold standard reference (n=2 per timepoint). miRge3.0 was used to quantify mature miRNA abundance in the STRS and matched small RNA-sequencing libraries (Methods). A strong correlation in the abundance of many of the most highly expressed miRNAs between STRS and small RNAseq was found (FIG. 8E, FIG. 9). Many examples of mature miRNA expression in STRS data were identified, including expression of classic “myomiRs”, miR-1a-3p, miR-133a/b-3p, and miR-206-3p (FIG. 8F). Consistent with previous studies, static expression of miR-1a-3p was detected across all four timepoints (FIG. 8D), whereas miR-206-3p was highly expressed within the injury locus five days post-injury, with very low levels of expression detected at other timepoints.

### Example 5. Spatial Total Transcriptomics Spatially Resolves Viral Infection of the Murine Heart

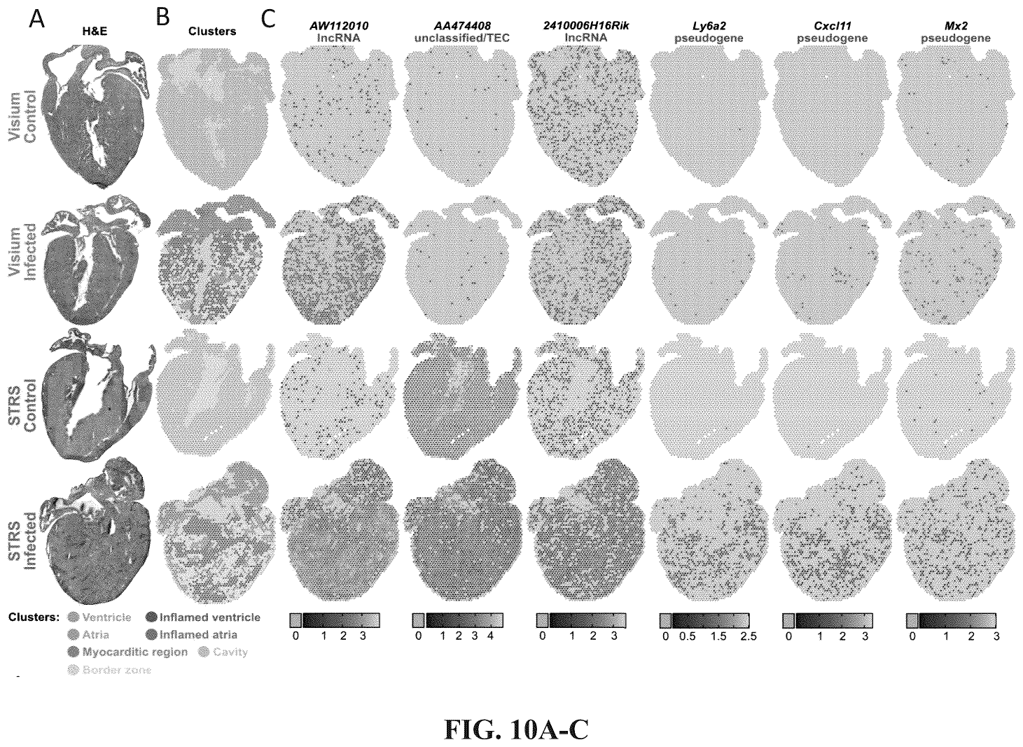

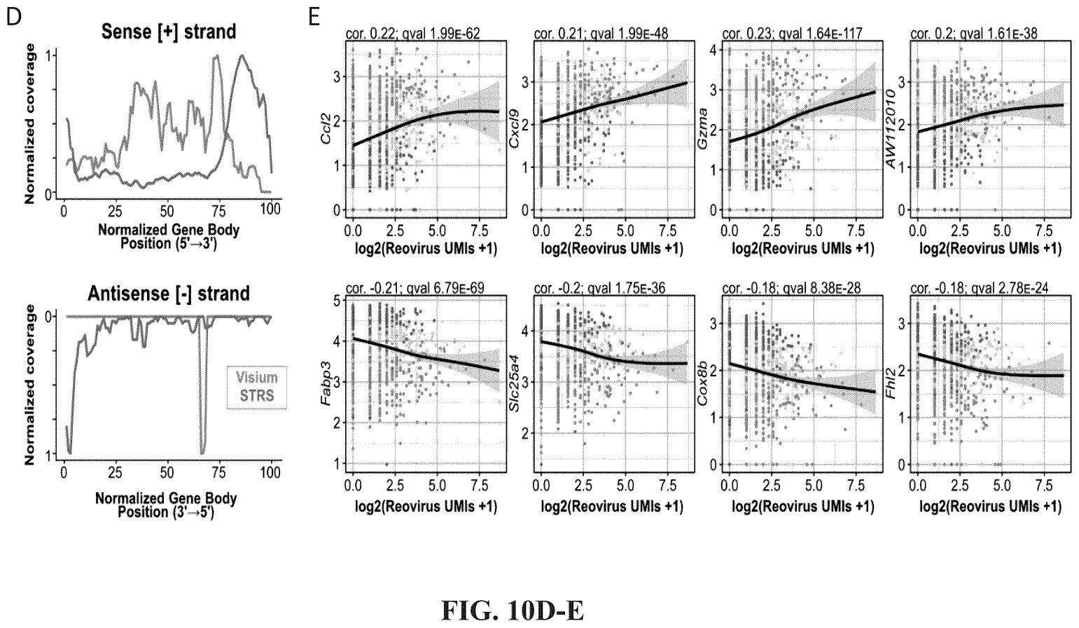

The potential for STRS to profile host-virus interactions in a mouse model of viral-induced myocarditis was then explored. Neonatal mice were orally infected with type 1-Lang reovirus (REOV), a double-stranded RNA virus with gene transcripts that are not polyadenylated. Within seven days of oral infection, REOV spreads to the heart and causes myocarditis. Visium and STRS were performed on hearts collected from REOV-infected and saline-injected control mice (FIG. 10A). We found that reovirus transcripts were only detected in the infected heart via STRS and that targeted enrichment of reovirus transcripts enabled deeper profiling of viral infection (FIG. 3D, FIG. 10A). Mapping these reads across the tissue revealed pervasive infection across the heart ( 1,329/2,501 or 53% spots under the tissue; FIG. 3D). Foci containing high viral UMI counts overlapped with the myocarditic regions as identified by histology.

Next the read coverage profiles across the ten REOV gene segments for REOV-enriched libraries from Visium and STRS samples were compared (FIG. 10D). As expected, STRS libraries had a peak in coverage at the 3′ end of viral gene segments. In contrast, the REOV-enriched Visium reads contained peaks in the middle of viral gene segments as expected for a chemistry that relies on the spurious capture of viral RNA at poly(A) repeats within the transcripts. Interestingly, STRS led to an overrepresentation of reads from the 5′ end of the sense [+] strand of all ten REOV segments. These reads may represent incomplete transcripts generated by transcriptional pausing of the REOV RNA polymerase or transcripts undergoing 3′ exonucleolytic degradation. Finally, the 3′ end of the antisense [−] strand for nine of the ten segments of the reovirus genome were detected, suggesting that STRS captures both strands of the dsRNA reovirus genome (FIG. 10D). These antisense reads were present at an average ratio of ˜1:40 compared to the sense reads. The current model for synthesis of reovirus dsRNA posits that dsRNA synthesis only occurs within a viral core particle after packaging of the ten viral positive-sense RNAs. Some possible explanations for the detection of the antisense strands. One is that detection of negative-strand viral RNA that is part of dsRNA that has been released from damaged viral particles either within the cytoplasm or within lysosomes is occurring. dsRNA released within endolysosomes can be transported into the cytoplasm by RNA transmembrane receptors SIDT1 and SIDT227,28. A second possibility is that antisense [−] viral RNA is synthesized prior to packaging of dsRNA into viral particles.

Because STRS efficiently recovers viral RNA, host transcriptomic responses with viral transcript counts for spots in inflamed regions could be directly correlated. Inflammation-associated cytokine transcripts such as Ccl2 and Cxcl9, and immune cell markers such as Gzma and Trbc2 to be upregulated in spots with high viral counts were found (FIG. 10E). Analysis continued by performing unsupervised clustering (FIG. 10B) and differential gene expression analysis to identify transcripts associated with infection which are more readily detected by STRS (FIG. 10C). AW112010, which has recently been shown to regulate inflammatory T cell states, was only found in infected samples and was more abundant in the STRS data compared to Visium. STRS also led to increased detection of putative protein-coding genes, including Ly6a2, Cxcl11, and Mx2, which were associated with infection. Interestingly, all three genes are annotated as pseudogenes in GENCODE annotations but have biotype conflicts with other databases. The increased abundance as measured by STRS could reflect differential mRNA polyadenylation for these transcripts. Overall, STRS enabled more robust analysis of the host response to infection by increasing the breadth of captured transcript types and by providing direct comparison with viral transcript abundance.

Further to the increased abundance measured by STRS in murine cardiac tissue, the STRS method was used with another commercially available RNA spatial mapping kit, the Seeker platform from Curio Bioscience. STRS-HD was performed using a modified version of the Seeker protocol. Sections (10 μm thick) from fresh frozen tissue blocks were mounted onto the Seeker 3×3 mm Tiles (Curio Bioscience). After sectioning, the Tiles were carefully placed into 300 μl of pre-chilled methanol and fixed for 30 min at −20° C. After fixation, Tiles were carefully removed from the methanol, placed into an empty 1.5 ml tube, and spun in a table-top centrifuge for 2 seconds to dry the tissue. Tiles were then transferred to a new 1.5 ml tube. In situ polyadenylation was then performed using yeast poly(A) polymerase (yPAP; Thermo Scientific, catalog no. 74225Z25KU). First, samples were equilibrated by adding 200 μl 1× wash buffer (40 μl 5×yPAP Reaction Buffer, 4 μl 40 U μl—1 Protector RNase Inhibitor, 156 μl nuclease-free H2O) (Protector RNase Inhibitor; Roche, catalog no. 3335402001) to each tube and incubating at room temperature for 30 s. The buffer was then removed. Next, 200 μl yPAP enzyme mix (40 μl 5×yPAP reaction buffer, 8 μl 600U/μl yPAP enzyme, 10 μl 10 mM ATP, 8 μl 40 U/μl Protector RNase Inhibitor, 134 μl nuclease-free H2O) was added to each reaction chamber. Tiles were then incubated at 37° C. for 25 min. The enzyme mix was then removed. After in situ polyadenylation, Tiles were transferred into 200 μl of 0.1% pepsin in 0.1M HCl and incubated at 37 for 15 min. Following permeabilization, the Tile was carefully transferred to 200 μl of Hybridization Buffer and the remaining steps in the standard Seeker protocol were followed. The libraries were then pooled and sequenced using a NextSeq 2000 (Illumina). Mice were orally infected with type 1-lang Reovirus, and heart tissues were collected seven days post-infection. (A) Spatial map showing the capture of RNAs which map to the host genome or to the reovirus genome. The top row shows the tissue processed with the standard Seeker workflow. The bottom row shows the tissue processed using STRS-HD. (B) Zoomed in view of a region with high reovirus capture.

### Example 7. Comparison of STRS-HD and Seeker Methodologies in the Testes

STRS-HD was performed using a modified version of the Seeker protocol. Sections (10 μm thick) from fresh frozen tissue blocks were mounted onto the Seeker 3×3 mm Tiles (Curio Bioscience). After sectioning, the Tiles were carefully placed into 300 μl of pre-chilled methanol and fixed for 30 min at −20° C. After fixation, Tiles were carefully removed from the methanol, placed into an empty 1.5 ml tube, and spun in a table-top centrifuge for 2 seconds to dry the tissue. Tiles were then transferred to a new 1.5 ml tube. In situ polyadenylation was then performed using yeast poly(A) polymerase (yPAP; Thermo Scientific, catalog no. 74225Z25KU). First, samples were equilibrated by adding 200 μl 1× wash buffer (40 μl 5×yPAP Reaction Buffer, 4 μl 40 U μl—1 Protector RNase Inhibitor, 156 μl nuclease-free H2O) (Protector RNase Inhibitor; Roche, catalog no. 3335402001) to each tube and incubating at room temperature for 30 s. The buffer was then removed. Next, 200 μl yPAP enzyme mix (40 μl 5×yPAP reaction buffer, 8 μl 600 U/μl yPAP enzyme, 10 μl 10 mM ATP, 8 μl 40 U/μl Protector RNase Inhibitor, 134 μl nuclease-free H2O) was added to each reaction chamber. Tiles were then incubated at 37° C. for 25 min. The enzyme mix was then removed. Before running STRS-HD, optimal tissue permeabilization time for heart was determined to be 15 min using the Visium Tissue Optimization Kit from 10× Genomics. The optimal permeabilization time for testes was found to be 10 min. After in situ polyadenylation, Tiles were transferred into 200 μl of 0.1% pepsin in 0.1M HCl and incubated at 37 for 15 min. Following permeabilization, the Tile was carefully transferred to 200 μl of Hybridization Buffer and the remaining steps in the standard Seeker protocol were followed. The libraries were then pooled and sequenced using a NextSeq 2000 (Illumina).

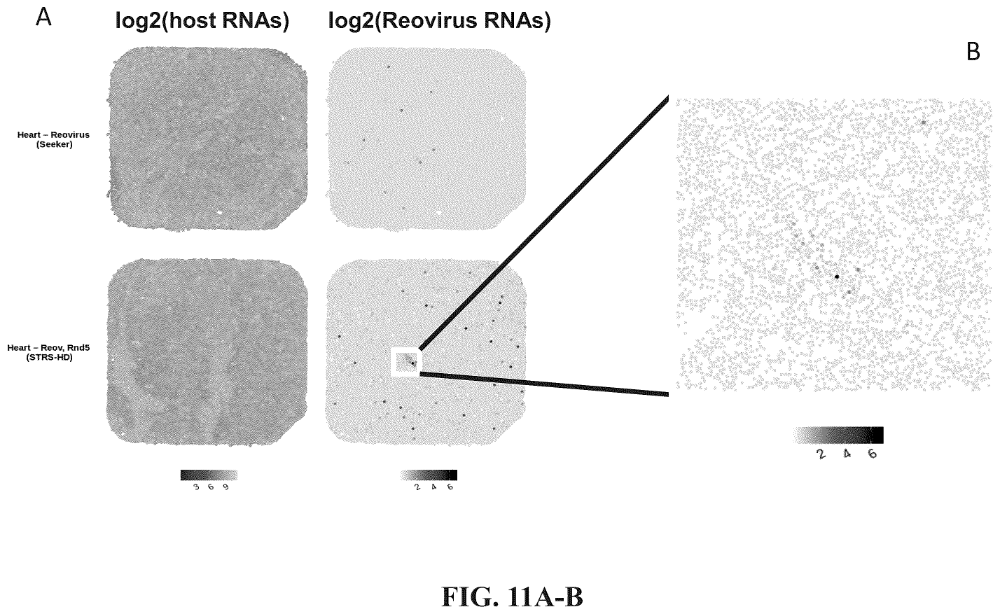

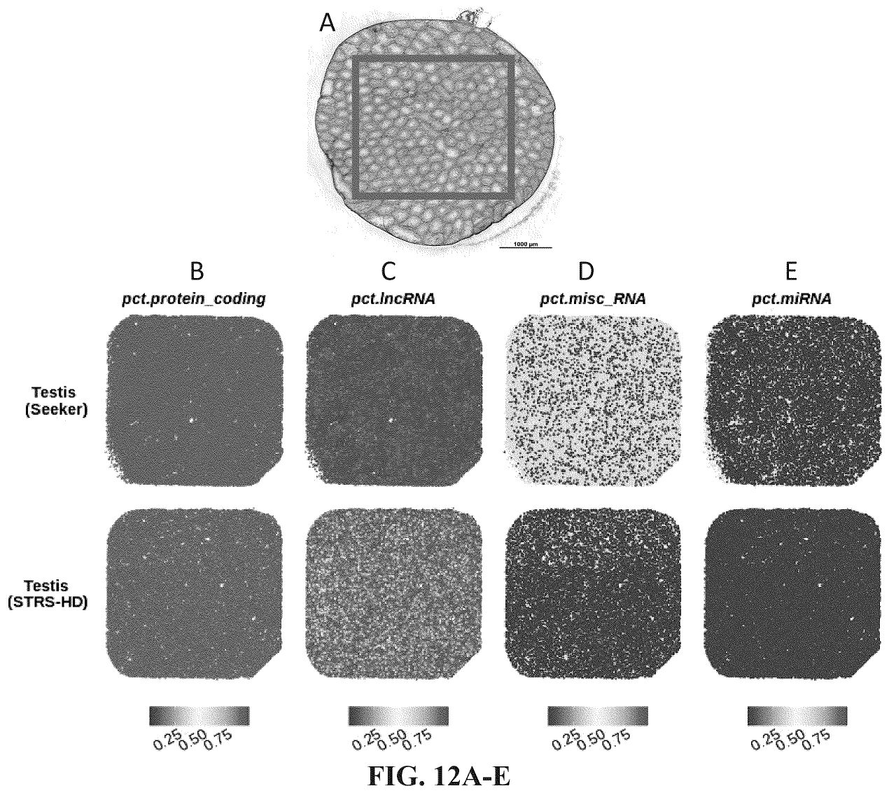

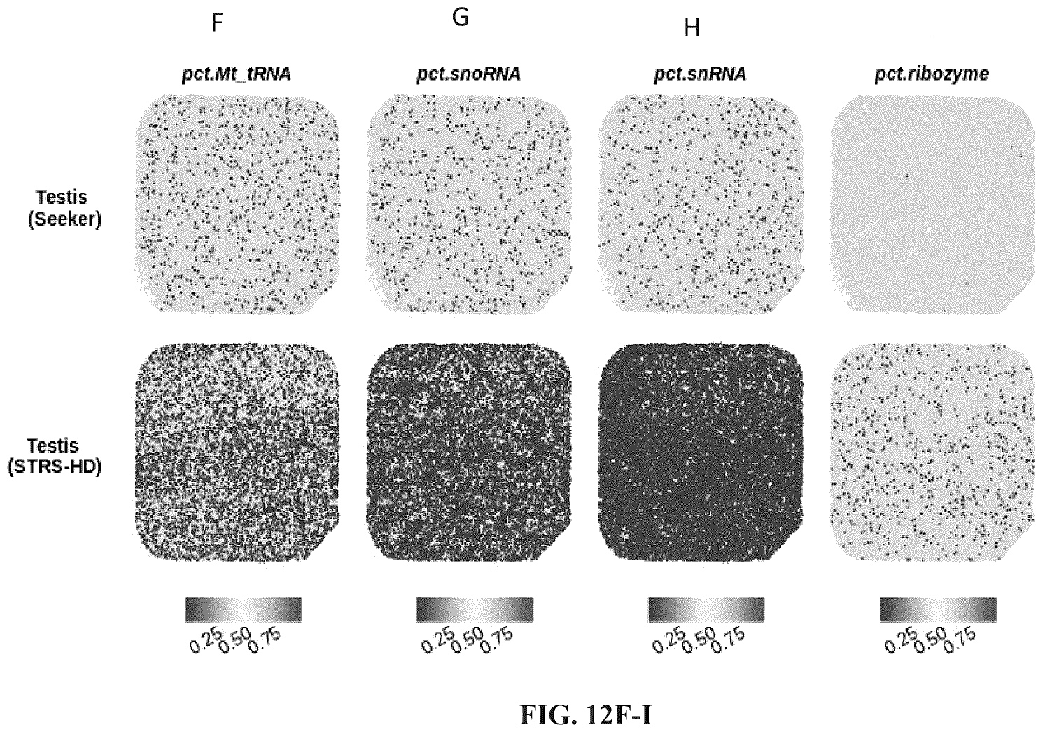

STRS-HD enabled robust detection of several types of noncoding RNAs which are poorly recovered or not detected at all by the Seeker method, including long non-coding RNAs (FIG. 12C), miscellaneous RNAs (FIG. 12D), microRNAs (FIG. 12E), transfer RNAs (FIG. 12F), small nucleolar RNAs (FIG. 12G), and ribosomal RNAs (FIG. 12I).

### Example 8. Tuning Poly(A) Tail Length via Biotin-11-ATP

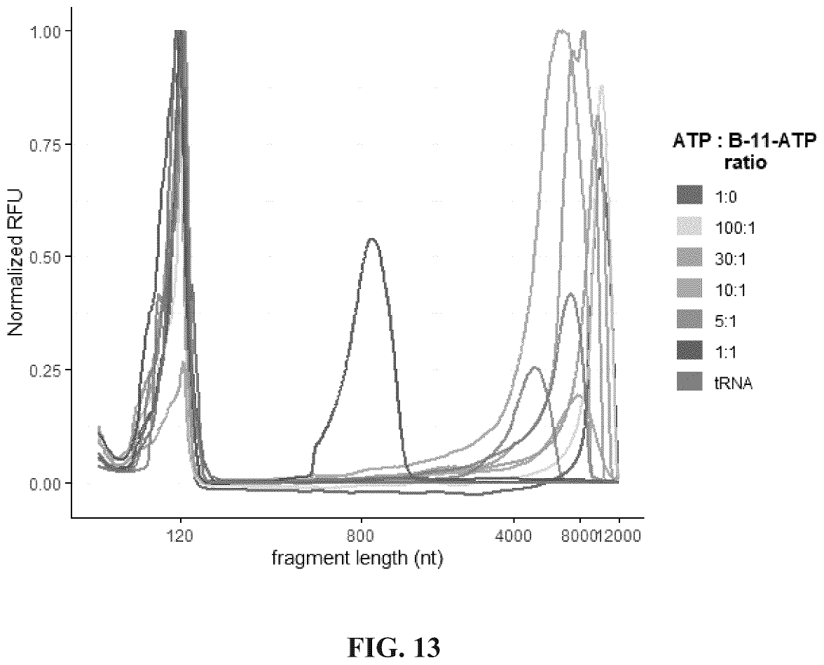

Purified transfer RNA (120 bp long, pink) was incubated with yeast poly(A) polymerase with varying ratios of ATP to biotin-11-ATP (B-11-ATP). Total concentration of ATP+B-11-ATP was held constant across experimental conditions. Reactions were performed to match the conditions of STRS. As is seen in FIG. 13, ratios of ATP to B-11-ATP were effecting in stopping the polyadenylation of noncoding RNA occurring through the yeast poly(A) polymerase. The x-axis shows the lengths of RNAs after polyadenylation, and the y-axis shows the abundance of RNAs, normalized by sample.

### Example 9. Spatial Total RNA-Sequencing (STRS) Improves Capture of Microbial Genera and RNAs in the Gut Microbiome

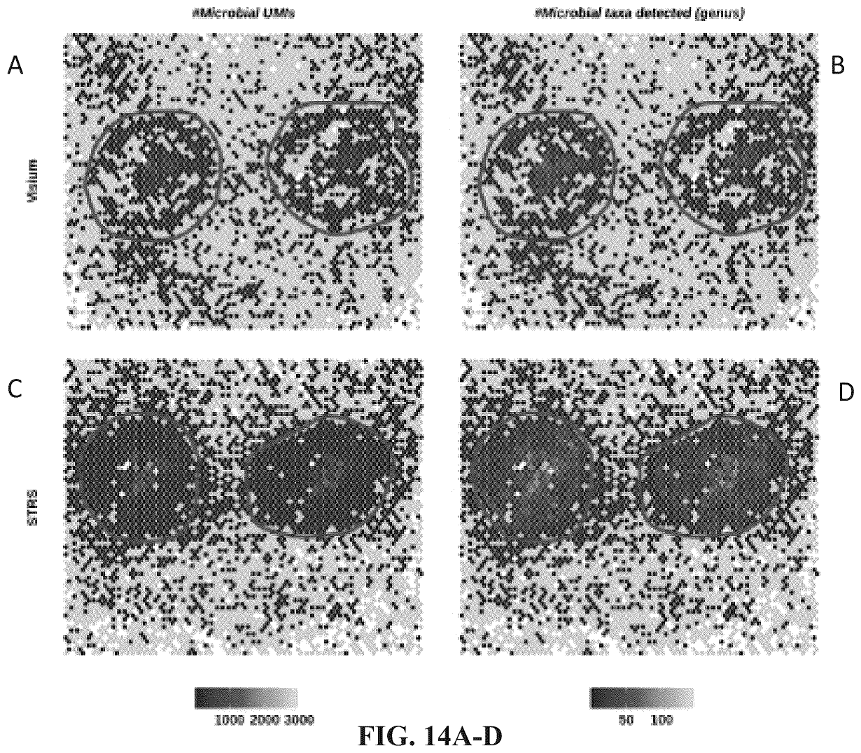

Fresh frozen mouse intestine tissue was cryosectioned at −20° C. to obtain 10 μm sections. The section was then fixed using Methacarn at room temperature for 15 minutes. The Methacarn solution consisted of 60% Absolute Methanol, 30% Chloroform, and 10% Glacial Acetic Acid. Next, the Visium H&E staining protocol was followed. The gut section on the slide was treated with isopropanol and allowed to dry, then incubated with hematoxylin, blueing buffer, and eosin. After imaging the sample, the STRS protocol was followed. The RNA polyadenylation step was carried out using Poly(A) Polymerase incubated for 25 minutes at 37° C. The polyadenylation step was necessary to capture the microbial transcripts along with other RNAs. An alternative protocol was also tested, which included a microbial cell-wall digestion step before polyadenylation; The tissue was rehydrated in a cell-wall permeabilization buffer (300 U/uL Lysozyme (ReadyLyse Lysozyme Biosearch Technologies), 10 mM Tris-HCl pH 7.5, NaCl 100 mM, 1 U/uL murine RNAse-inhibitor (NEB)) at room temperature for 30 minutes before the polyadenylation step. The STRS protocol was shown to be compatible with microbial transcript capture. Results are shown in the spatial maps of FIG. 14, where the number of unique molecules (UMIs) that map to microbial genomes are detected in each spot (FIG. 14A and FIG. 14C) and the number of total microbial taxa are detected in each spot (FIG. 14B and FIG. 14D).

### General Methods

The Cornell University Institutional Animal Care and Use Committee (IACUC) approved all animal protocols, and experiments were performed incompliance with its institutional guidelines. For skeletal muscle samples, adult female C57BL/6J mice were obtained from Jackson Laboratories (#000664; Bar Harbor, ME) and were used at 6 months of age. For heart samples, confirmed pregnant female C57BL/6J mice were ordered from Jackson Laboratories to be delivered at embryonic stage E14.5.

### Viral Infection

Litters weighing 3 gram/pup were orally gavaged using intramedic tubing (Becton Dickinson Cat #427401 with 50 μl with 107 PFU reovirus type 1-lang (TIL) strain in 1× phosphate buffered saline (PBS) containing green food color (McCormick) via a 1 ml tuberculin slip tip syringe (BD 309659) and 30G×½ needle (BD Cat #305106). Litters treated with 1×PBS containing green food color alone on the same day were used as mock controls for the respective infection groups. The mock-infected and reovirus-infected pups were monitored and weighed daily until the time points used in the study (7 days post infection). After dissection, samples were embedded in O.C.T. Compound (Tissue-Tek) and frozen fresh in liquid nitrogen.

### Muscle Injury

To induce muscle injury, both tibialis anterior muscles of old (20 months) C57BL/6J mice were injected with 10 μl of notexin (10 μg/ml; Latoxan; France). Either before injury or 2-, 5-, or 7-days post-injury (dpi), mice were sacrificed and tibialis anterior muscles were collected. After dissection, samples were embedded in O.C.T. Compound (Tissue-Tek) and frozen fresh in liquid nitrogen.

### In Situ Polyadenylation and Spatial Total RNA-Sequencing (STRS)

Spatial total RNA-sequencing was performed using a modified version of the Visium protocol. 10 um thick tissue sections were mounted onto the Visium Spatial Gene Expression v1 slides. For heart samples, one tissue section was placed into each 6×6 mm capture area. For skeletal muscle samples, two tibialis anterior sections were placed into each capture area. After sectioning, tissue sections were fixed in methanol for 20 minutes at −20° C. Next, H&E staining was performed according to the Visium protocol, and tissue sections were imaged on a Zeiss Axio Observer Z1 Microscope using a Zeiss Axiocam 305 color camera. H&E images were shading corrected, stitched, rotated, thresholded, and exported as TIFF files using Zen 3.1 software (Blue edition). After imaging, the slide was placed into the Visium Slide Cassette. In situ polyadenylation was then performed using yeast Poly(A) Polymerase (yPAP; Thermo Scientific, Cat #74225Z25KU). First, samples were equilibrated by adding 100 μl of 1× wash buffer (20 μl 5×yPAP Reaction Buffer, 2 μl 40U/μl Protector RNase Inhibitor, 78 μl nuclease-free H2O) to each capture area and incubating at room temperature for 30 seconds. The buffer was then removed. Next, 75 μl of yPAP enzyme mix (15 μl 5×yPAP Reaction Buffer, 3 μl of 600 U/μl yPAP enzyme, 1.5 μl 25 mM ATP, 3 μl 40U/μl Protector RNase Inhibitor, 52.5 μl nuclease-free H2O) was added to each reaction chamber. STRS was also tested with 20 U/μl of SUPERase-In RNase-Inhibitor, but we found that SUPERase was not able to prevent degradation of longer transcripts during in situ polyadenylation (FIGS. 6c-d). The reaction chambers were then sealed, and the slide cassette was incubated at 37° C. for 25 minutes. The enzyme mix was then removed. Prior to running STRS, optimal tissue permeabilization time for both heart and skeletal muscle samples was determined to be 15 minutes using the Visium Tissue Optimization Kit from 10× Genomics. The optimal permeabilization time for testes was found to be 10 min. Following in situ polyadenylation, the standard Visium library preparation was followed to generate cDNA and final sequencing libraries. The libraries were then pooled and sequenced according to guidelines in the Visium Spatial Gene Expression protocol using either a NextSeq 500 or NextSeq 2000 (Illumina, San Diego, CA).

### Small RNA-Sequencing

For skeletal muscle samples, following the injury time course, tibialis anterior muscles were dissected and snap frozen with liquid nitrogen. The Norgen Total RNA Purification Kit (Cat. 17200) was used to extract RNA from 10 mg of tissue for each sample. For heart samples, following the infection time course, hearts were dissected, embedded in OCT, and frozen in liquid nitrogen. RNA was extracted with Trizol (Invitrogen, Cat. 15596026) and glycogen precipitation for a small fraction of each of the heart samples. RNA quality was assessed via High Sensitivity RNA ScreenTape Analysis (Agilent, Cat. 5067-5579) and all samples had RNA integrity numbers greater than or equal to 7.

Small RNA sequencing was performed at the Genome Sequencing Facility of Greehey Children's Cancer Research Institute at the University of Texas Health Science Center at San Antonio. Libraries were prepared using the TriLink CleanTag Small RNA Ligation kit (TriLink Biotechnologies, San Diego, CA). Libraries were sequenced with single-end 50× using a HiSeq2500 (Illumina, San Diego, CA).

### Preprocessing and Alignment of Spatial Total RNA-Sequencing Data

All code used to process and analyze these data can be found at https://github.com/mckellardw/STRS.

Reads were first trimmed using cutadapt v3.4 to remove the following sequences: 1) poly(A) sequences from the three prime ends of reads, 2) the template switch oligonucleotide sequence from the five prime end of reads which are derived from the Visium Gene Expression kit (sequence: CCCATGTACTCTGCGTTGATACCACTGCTT; SEQ ID NO: 1), 3) poly(G) artifacts from the three prime ends of reads, which are produced by the Illumina two-color sequencing chemistry when cDNA molecules are shorter than the final read length, and 4) the reverse complement of the template switching oligonucleotide sequence from the five prime ends of reads (sequence: AAGCAGTGGTATCAACGCAGAGTACATGGG; SEQ ID NO: 2). Next, reads were aligned using either STAR v2.7.10a or kallisto v0.48.0. Workflows were written using Snakemake v6.1.0.

For STAR, the genomic reference was generated from the GRCm39 reference sequence using GENCODE M28 annotations. For STAR alignment, the following parameters, based on work by Isakova et al, were used: outFilterMismatchNoverLmax=0.05, outFilterMatchNmin=16, outFilterScoreMinOverLread=0, outFilterMatchNminOverLread=0, outFilterMultimapNmax=50. Aligned reads were deduplicated for visualization using umi-tools v1.1.242.

For kallisto, a transcriptomic reference was also generated using the GRCm39 reference sequence and GENCODE M28 annotations. The default k-mer length of 31 was used to generate the kallisto reference. Reads were pseudoaligned using the ‘kallisto bus’ command with the chemistry set to “VISIUM” and the ‘fr-stranded’ flag activated to enable strand-aware quantification. Pseudoaligned reads were then quantified using bustools v0.41.0. First, spot barcodes were corrected with ‘bustools correct’ using the “Visium-v1” whitelist provided in the Space Ranger software from 10× Genomics. Next, the output bus file was sorted and counted using ‘bustools sort’ and ‘bustools count’, respectively. To estimate the number of spliced and unspliced transcripts, reads pseudoaligned using kb-python v0.26.0, using the “lemanno” workflow.

Spots were manually selected based on the H&E images using Loupe Browser from 10× Genomics. Spatial locations for each spot were assigned using the Visium coordinates provided for each spot barcode by 10× Genomics in the Space Ranger software (“Visium-v1_coordinates.txt”). Downstream analyses with the output count matrices were then performed using Seurat v4.0.4. In addition to manual selection, spots containing fewer than 500 detected features or fewer than 1000 unique molecules were removed from the analysis. Counts from multimapping features were collapsed into a single feature to simplify quantification.

Mature microRNA Quantification

For STRS data: after trimming (see above), barcode correction with STAR v2.7.10a, and UMI-aware deduplication with umi-tools v1.1.2, reads were split across all 4992 spot barcodes and analyzed using miRge3.0 v0.0.920. Reads were aligned to the miRbase reference provided by the miRge3.0 authors. MiRNA counts were log-normalized according to the total number of counts detected by kallisto and scaled using a scaling factor of 1000. For small RNAseq data: Reads were first trimmed using trim_galore v0.6.5. Reads were then aligned and counted using miRge3.0 v0.0.9.

### Unsupervised Clustering and Differential Gene Expression Analysis of Spot Transcriptomes

Spot UMI counts as generated by kallisto were used. First, counts were log-normalized and scaled using default parameters with Seurat. Principal component analysis was then performed on the top 2000 most variable features for each tissue slice individually. Finally, unsupervised clustering was performed using the ‘FindClusters( )’ function from Seurat. The top principal components which accounted for 95% of variance within the data were used for clustering. For skeletal muscle samples, a clustering resolution was set to 0.8. For heart samples, clustering resolution was set to 1.0. Default options were used for all other parameters. Finally, clusters were merged according to similar gene expression patterns and based on histology of the tissue under each subcluster.

Differential gene expression analysis was performed using the ‘FindAllMarkers( )’ function from Seurat. Default parameters were used, including the use of the Wilcoxon ranked sum test to identify differentially expressed genes. To identify features enriched in the skeletal muscle STRS datasets, all Visium and STRS were first merged and compared according to the method used (Visium vs. STRS). To identify cluster-specific gene expression patterns, skeletal muscle samples were first clustered as described above individually. STRS samples were then merged, and differential gene expression analysis was performed across the three injury region groups.

### Targeted Pulldown Enrichment of Viral Fragments

Hybridization-based enrichment of viral fragments was performed on the Visium and STRS libraries for reovirus-infected hearts using the xGen Hybridization and Wash Kit (IDT; 1080577). In this approach, a panel of 5′-biotinylated oligonucleotides was used for capture and pulldown of target molecules of interest, which were then PCR amplified and sequenced. A panel of 202 biotinylated probes tiled across the entire reovirus TIL genome was designed to selectively sequence viral molecules from the sequencing libraries. After fragmentation and indexing of cDNA, 300 ng of the final Visium or STRS sequencing libraries from reovirus-infected hearts were used for xGen hybridization capture using the xGen NGS Target Enrichment Kit protocol provided by the manufacturer. One round of hybridization capture was performed for the STRS library followed by 14 cycles of PCR amplification. Because of the reduced number of captured molecules, two rounds of hybridization were performed on the Visium libraries. Enriched VIsium libraries were PCR-amplified for 18 cycles after the first round of hybridization and by 5 cycles after the second round of hybridization. Post-enrichment products were pooled and sequenced on the Illumina NextSeq 500.

### Correlation Analysis Between Reovirus Counts and Host Gene Expression

A generative additive model (GAM) implemented in Monocle v2.18.045 was used to find genes that vary with viral UMI count. A Seurat object for STRS data and viral UMI counts from the reovirus-infected heart was converted to a CellDataSet object using the ‘as.CellDataSet( )’ command implemented in Seurat. The expression family was set to “negative binomial” as suggested for UMI count data in the Monocle documentation. The CellDataSet object was then preprocessed to estimate size factors and dispersion for all genes. Genes expressed in fewer than 10 spots were removed. Within the remaining genes, we then used the GAM implemented in the ‘differentialGeneTest( )’ command in Monocle to identify genes that vary with log-transformed viral UMI counts. To find the direction in which these genes varied with viral UMI counts, the Pearson correlation was calculated for all genes with log-transformed viral UMI counts.

### Data and Code Availability

Previously published spatial RNA-sequencing data were downloaded from Gene Expression Omnibus (GEO) and are available under the following accession numbers; regenerating skeletal muscle5 GSE161318, infected heart tissue4 GSE189636. Spatial Total RNA-Sequencing data generated in this study can be found on GEO under the accession number GSE200481. Small RNA-sequencing data are available on GEO under the accession number GSE200480 A detailed protocol for performing STRS as well as custom analysis scripts for aligning and processing STRS data can be found at https://github.com/mckellardw/STRS.

What is claimed is:

1. A method for spatial detection of RNA molecules in a biological sample, comprising:
(a) providing a substrate defined by an array of spots, wherein each spot comprises DNA oligomers immobilized thereto, wherein each of the DNA oligomers comprises:
(i) a spatial barcode, wherein all DNA oligomers in one spot share the same spatial barcode, which is different from the spatial barcodes in other spots; and
(ii) a poly(dT) sequence;
(b) placing a biological sample onto the substrate;
(c) contacting the substrate with a Poly(A) polymerase enzyme mix comprising a Poly(A) polymerase and a polymerase reaction buffer reagent to perform in situ polyadenylation;
(d) capturing RNA molecules from the biological sample;
(e) adding reverse transcription reagents to generate cDNA molecules from captured RNA molecules, wherein cDNA molecules generated from RNAs captured by DNA oligomers on a spot comprise a spatial barcode common to the spot; and
(f) obtaining a map of spatial gene expression based on the cDNA molecules generated.

2. The method of claim 1, wherein step (d) includes permeabilizing the cells in the biological sample to permit release and capture of the RNA molecules from the cells in the biological sample.

3. The method of claim 1 or 2, wherein each of the DNA oligomers comprises:
an oligonucleotide sequence; and/or
a unique molecular identifier sequence.

4. The method according to any one of the proceeding claims, wherein step (b) further comprises fixing the biological sample (e.g., using formaldehyde, Formalin-fixed, parafin-embedded (FFPE), Acetone, Methanol+acetone, Glyoxal fixation).

5. The method of claim 4, wherein step (b) further comprises staining the fixed biological sample.

6. The method of claim 5, wherein step (b) further comprises capturing an image of the fixed and stained biological sample.

7. The method according to any one of the preceding claims, wherein the sequences of the generated cDNA are obtained.

8. The method of claim 7, wherein the generated cDNAs with spatial barcodes and the cDNA sequences are used to map the spatial gene expression.

9. The method of claim 7, wherein the generated cDNAs and the cDNA sequences are correlated with the captured image of the fixed and stained biological sample to map the spatial gene expression.

10. The method according to any one of the preceding claims, wherein step (e) further comprises initiating second strand synthesis via the addition of a second strand primer.

11. The method according to any one of the proceeding claims, wherein the cDNAs are denatured and transferred from the spots to a solution readily usable for amplification.

12. The method of claim 11, wherein the amplified cDNAs are further processed for optimal amplicon size.

13. The method according to any one of the preceding claims, wherein step (c) further comprises, prior to the contacting step, equilibrating the substrate by adding a wash buffer comprising Poly(A) polymerase reaction buffer, an RNase inhibitor, and nuclease free water to the substrate.

14. The method of claim 13, wherein step (c) comprises, after the equilibrating, adding a Poly(A) polymerase enzyme mix which comprises Poly(A) polymerase reaction buffer, a Poly(A) polymerase enzyme, adenosine triphosphate (ATP), RNase inhibitor, and nuclease-free water and incubating.

15. The method according to any one of the preceding claims, wherein the Poly(A) polymerase enzyme is a yeast Poly(A) polymerase (such as Thermo Scientific, cat #74225Z25KU, or an E. coli Poly(A) polymerase).

16. The method according to any one of the preceding claims, wherein the substrate is composed of a material selected from the group consisting of glass, silicon, poly-L-lysine coated material, nitrocellulose, polystyrene, cyclic olefin copolymers (COCs), cyclic olefin polymers (COPs), polypropylene, polyethylene and polycarbonate.

17. The method according to any one of the preceding claims, wherein the array of spots comprises 10-100,000,000 spots, such as at least 10, at least 100, at least 1,000, at least 10,000, at least 50,000, at least 100,000, at least 200,000, at least 300,000, at least 400,000, at least 500,000, at least 1,000,000, at least 2,000,000, at least 10,000,000, at least 20,000,000, at least 30,000,000, at least 40,000,000, at least 50,000,000, at least 75,000,000, or at least 100,000,000 spots.

18. The method according to any one of the preceding claims, wherein the array is placed within a capture area having a dimension of about 1 mm² to about 100 mm².

19. The method claim 18, wherein the capture area has a dimension of up to 10 mm².

20. The method claim 18, wherein the capture area has a dimension of up to 6.5 mm².

21. The method according to claim 18, wherein the capture area has a dimension of up to 3 mm².

22. The method according to any one of the preceding claims, wherein the spots are about 10 nm to about 1 mm in diameter.

23. The method according to any one of the preceding claims, wherein the spots are about 500 nm to about 125 μm apart as measured by center of spot to center of spot.

24. The method according to any one of the preceding claims, wherein the spots are less than 60 μm in diameter and are no more than 100 μm apart as measured by center of spot to center of spot.

25. The method according to any one of the preceding claims, wherein the spots are less than 220 nm in diameter and are no more than 750 nm apart as measured by center of spot to center of spot.

26. The method of any one of the preceding claims, wherein the spots are in an organized pattern in the array.

27. The method of any one of claims 1-25, wherein the spots are randomly distributed in the array.

28. The method of any one of the preceding claims, wherein the spatial resolution is from about 1 micron to about 100 microns.

29. The method of either claim 24 or 28, wherein the spatial resolution is about 60 microns.

30. The method of either claim 25 or 28, wherein the spatial resolution is about 10 microns.

31. The method according to any one of the proceeding claims, wherein the length of the poly(A) tail is controlled in the in situ polyadenylation.

32. The method according to claim 31, wherein the length of the poly(A) tail is about 10 base pairs to about 4,000 base pairs.

33. The method according to either claim 31 or 32, wherein the length of the poly(A) tail is less than about 2,000 base pairs.

34. The method according to any one of claims 31 to 33, wherein the length of the poly(A) tail is less than about 1,600 base pairs.

35. The method according to any one of claims 31 to 34, wherein the length of the poly(A) tail is less than about 1,000 base pairs.

36. The method according to any one of claims 31-35, wherein the Poly(A) polymerase enzyme mix includes (i) ATP and (ii) biotin-11-ATP or dATP at a ratio of at least 5:1.

37. The method of claim 36, wherein the ratio of ATP to biotin-11-ATP or dATP is 1:1.

38. The method according to any one of the proceeding claims, wherein the poly(dT) sequence comprises a VN sequence at the 3′ end wherein the V is any nucleotide base other than T and N is any nucleotide base.

39. The method according to any one of the preceding claims, wherein the biological sample is a tissue.

40. The method of claim 39, wherein the tissue is selected from the group of connective tissue, epithelial tissue, muscle tissue, and nervous tissue.

41. The method of claim 40, wherein the muscle tissue is a cardiac, skeletal, or smooth muscle tissue.

42. The method of claim 40, wherein the epithelial tissue is simple squamous, stratified squamous, simple cuboidal, stratified cuboidal, simple columnar, stratified columnar, pseudostratified columnar, or transitional epithelia.

43. The method of claim 40, wherein the connective tissue is connective tissue proper or specialized connective tissue.

44. The method of claim 43, wherein the connective tissue proper is loose or dense tissue, comprising collagen, reticular, or elastic fibers.

45. The method of claim 43, wherein the specialized connective tissue comprises adipose, cartilage, bone, blood, reticular, and lymphatic tissues

46. The method of claim 39, wherein the biological sample is a combination of tissue types which form an organ.

47. The method of claim 46, wherein the biological sample is taken from a testis.

48. The method according to any one of the preceding claims, wherein the biological sample is a histological section of tissue.

49. The method according to any one of the preceding claims, wherein the RNAs captured are ribonucleic acids (RNAs), RNA degradation products, RNAs comprising a poly(A) tail, messenger RNA (mRNA), long noncoding RNAs (lncRNAs), long intergenic noncoding RNAs (lincRNAs), cis-natural antisense transcripts (cisNATs), antisense RNAs, ribosomal RNAs (TRNAs), microRNAs (miRNAs), small interfering RNAs (siRNAs), short hairpin RNAs (shRNAs), guide RNAs (gRNAs), transfer RNAs (tRNAs), small nuclear RNAs (snRNAs), small nucleolar RNAs (snoRNAs), small Cajal body-specific RNA (scaRNAs), enhancer RNAs (eRNAs), piwi-interacting RNAs (piRNAs), Y RNAs, non-coding RNAs, vault RNA, viral RNA, microbial RNA such as bacterial RNA, archaeal RNA, or fungal RNA, or combinations thereof.

50. The method of claim 48, wherein the RNAs captured are viral RNA, bacterial RNA, archaeal RNA, fungal RNA, or a combination thereof.

51. The method of claim 1, wherein further comprises isolating a subpopulation of cDNAs from the cDNAs generated in step (e).

52. The method of claim 51, wherein the subpopulation of cDNAs is generated from viral RNAs, bacterial RNA, archaeal RNA, or fungal RNA.

53. The method of claim 51 or 52, further comprising obtaining the sequences of the cDNAs in the isolated subpopulation.

54. The method according to any one of the preceding claims, wherein the biological sample is a tissue sample of an injured tissue or an organ suspected to suffer an infection.

55. The method of claim 54, wherein the tissue sample is a tumor section, gut microbiome, brain section, patient biopsy, or a plant sample.

56. The method of claim 54 or 55, further comprising comparing the spatial gene expression map of the tissue sample to (i) the spatial gene expression map of a control sample, or (ii) the spatial gene expression map of another sample of the same tissue taken at a different time point.

57. A kit comprising:
a substrate defined by an array of spots, wherein each spot comprises DNA oligomers (for capturing and priming of polyadenylated RNAs) immobilized on the substrate, wherein each of the DNA oligomers comprises:
(i) a spatial barcode, wherein all primers in one spot share the same spatial barcode,
which is different from the spatial barcodes in other spots; and
(ii) a poly(dT) sequence;
at least one reagent comprising a Poly(A) polymerase enzyme mix; and
optionally instructions for use.

58. The kit of claim 57, wherein each of the DNA oligomers further comprises:
an oligonucleotide sequence; and/or
a unique molecular identifier sequence.

59. The kit of claim 57 or 58, wherein the Poly(A) polymerase enzyme mix comprises:
a polymerase reaction buffer reagent;
a poly(A) polymerase enzyme reagent; and optionally
nuclease free water reagent.

60. The kit of according to any one of claims 57-59, wherein the Poly(A) polymerase enzyme mix further comprises adenosine triphosphate reagent and/or an RNase inhibitor reagent.

61. The kit according to any one of claims 57-60, wherein the kit further comprises a wash buffer reagent.

62. The kit of claim 61, wherein the wash buffer reagent comprises:
a polymerase reaction buffer reagent,
an RNase inhibitor reagent; and optionally
a nuclease-free water reagent.

63. The kit of claim 60, wherein the Poly(A) polymerase enzyme mix comprises (i) ATP and (ii) biotin-11-ATP or dATP, optionally at a ratio that is greater than about 5:1 ATP to biotin-11-ATP or dATP.

64. The kit according to any one of claims 57-63, wherein the poly(dT) sequence comprises a VN sequence at the 3′ end wherein the V is any nucleotide base other than T and N is any nucleotide base.

65. The kit according to any one of claims 57-64, wherein the reagents are either ready to use, concentrated, or a combination of ready to use and concentrated.

66. The kit according to any one of claims 57-65, wherein the reagents are provided in separate containers or provided in pre-mixed quantities of any combination of reagents.

67. The kit according to any one of claims 57-66, wherein the array of spots comprises 10-100,000,000 spots, such as at least 10, at least 100, at least 1,000, at least 10,000, at least 50,000, at least 100,000, at least 200,000, at least 300,000, at least 400,000, at least 500,000, at least 1,000,000, at least 2,000,000, at least 5,000,000, at least 10,000,000, at least 20,000,000, at least 30,000,000, at least 40,000,000, at least 50,000,000, at least 75,000,000, or at least 100,000,000 spots.

68. The kit according to any one of claims 57-67, wherein the array is placed within a capture area having a dimension of about 1 mm² to about 100 mm².

69. The kit according to claim 68, wherein the capture area has a dimension of up to 10 mm².

70. The kit according to claim 68, wherein the capture area has a dimension of up to 6.5 mm².

71. The kit according to claim 68, wherein the capture area has a dimension of up to 3 mm².

72. The kit according to any one of claims 57-71, wherein the spots are about 10 nm to about 1 mm in diameter.

73. The kit according to any one of claims 57-72, wherein the spots are about 20 μm to about 125 μm apart as measured by center of spot to center of spot.

74. The kit according to any one of claims 57-73, wherein the spots are less than 60 μm in diameter and are no more than 100 μm apart as measured by center of spot to center of spot.

75. The kit according to any one of claims 57-74, wherein the spots are less than 220 nm in diameter and are not more than 750 nm apart as measured by center of spot to center of spot.

76. The kit according to any one of claims 57-75, wherein the spots are in an organized pattern in the array.

77. The kit according to any one of claims 57-75, wherein the spots are randomly distributed in the array.

78. The kit according to any one of claims 57-77, wherein the spatial resolution ranges from about 1 micron to about 100 microns.

79. The kit according to either claim 74 or 78, wherein the spatial resolution is about 60 microns.

80. The kit according to either claim 75 or 78, wherein the spatial resolution is about 10 microns.


SEQUENCE LISTING (2 sequences)

SEQ ID NO: 1 - DNA, 30 bp, synthetic construct
  misc_feature 1..30: note = template switch oligonucleotidenucleic acid
  source 1..30: mol_type = other DNA
cccatgtactctgcgttgataccactgctt

SEQ ID NO: 2 - DNA, 30 bp, synthetic construct
  misc_feature 1..30: note = reverse complement of the template switching oligonucleotidenucleic acid
  source 1..30: mol_type = other DNA
aagcagtggtatcaacgcagagtacatggg